(19) United States
(12) Patent Application Publication
(10) Pub. No.: US 2021/0401798 A1
(43) Pub. Date: Dec. 30, 2021

(54) LIPOXIN A4 ANALOGS AND USES THEREOF

(71) Applicants: The Brigham and Women's Hospital, Inc., Boston, MA (US); Corbus Pharmaceuticals, Inc., Norwood, MA (US)

(72) Inventors: Nan CHIANG, Somerville, MA (US); Sheldon N. CRANE, Pincourt (CA); Thomas JENNEQUIN, Montreal (CA); Valdas JURKAUSKAS, Cambridge, MA (US); Clifton D. LEIGH, Somerville, MA (US); Kristos Adrian MOSHOS, Watertown, MA (US); Charles N. Serhan, Needham, MA (US); Mark A. Tepper, Newton, MA (US); Yuhua George Zhang, Auburndale, MA (US)

(73) Assignees: The Brigham and Women's Hospital, Inc., Boston, MA (US); Corbus Pharmaceuticals, Inc., Norwood, MA (US); Corbus Pharmaceuticals, Inc., Norwood, MA (US)

(21) Appl. No.: 17/287,691

(22) PCT Filed: Oct. 23, 2019
(86) PCT No.: PCT/US2019/057641

§ 371 Date: Apr. 22, 2021

Related U.S. Application Data
(60) Provisional application No. 62/749,335, filed on Oct. 23, 2018.

(51) Int. Cl. A61K 31/366; A61K 45/06

(52) U.S. Cl. CPC A61K 31/366; A61K 45/06

(57) ABSTRACT

The invention relates to (S)-6-((1 R,2E,4E,8E,10S)-11-(4-fluorophenoxy)-1,10-dihydroxyundeca-2,4,8-trien-6-yn-1-yl)-1,4-dioxan-2-one (compound (1)), which is a lactone-containing analog of lipoxin A₄ (LXA₄). In particular, the invention features pharmaceutical compositions including compound (1) and the use of compound (1) for the treatment of a disorder (e.g., a fibrotic disorder or an inflammatory disorder, such as an autoimmune disorder) in a subject in need thereof.

### BACKGROUND

Lipoxins (LXs), an acronym for lipoxygenase interaction products, are bioactive autacoid metabolites of arachidonic acid made by various cell types. Structurally, lipoxins are arachidonic acid metabolites that contain three hydroxyl groups and four double bonds. They are categorized as non-classic eicosanoids and members of the specialized pro-resolving mediators (SPMs) family of polyunsaturated fatty acid (PUFA) derived bioactive mediators. Like other SPMs, lipoxins form during, and then act to resolve, inflammatory responses. Initially, two lipoxins were identified, lipoxin Aa (LXA₄) and lipoxin B₄ (LXB₄), and more recent studies have identified epimers of these two lipoxins (e.g. 15-epi-LXA₄ and 15-epi-LXB₄).

LXA₄ and 15-epi-LXA₄ activate the G-protein coupled receptor ALX/FPR2. LXA₄ and 15-epi-LXA₄ are known to inhibit chemotaxis, transmigration, superoxide generation, NF-κB activation, and/or generation of pro-inflammatory cytokines (e.g. IL8, IL13, IL12, and IL5) by neutrophils, eosinophils, monocytes, innate lymphoid cells and/or macrophages, as well as suppress proliferation and production of IgM and IgG antibodies by B lymphocytes. These actions appear to involve stimulating anti-inflammatory signaling pathways, but also blocking the actions of other ALX/FPR2 ligands which simulate pro-inflammatory pathways.

Furthermore, LXA₄ was recently shown to be an endogenous allosteric modulator of the CB₁ cannabinoid receptor. LXA₄ enhances the affinity of anandamide (AEA) at this receptor to exert cannabimimetic effects in the brain by allosterically enhancing AEA signaling and thereby potentiating the effects of this endocannabinoid.

In vivo production of lipoxins is observed in many human diseases, including asthma, glomerulonephritis, and rheumatoid arthritis. Lipoxins have been shown to regulate polymorphonuclear leukocytes (PMN), chemotaxis, adhesion, and transmigration. Lipoxins are produced at the site of inflammation, and this production is coincident with the resolution of inflammation. This observation is consistent with a biological role for lipoxins in the resolution of inflammation. Lipoxins, including epi-lipoxins and analogs thereof, have been investigated for the treatment of disease, including inflammatory disorders.

Lipoxins are rapidly metabolized either by oxidation at C15 or reduction of the C13-C14 double bond. 15-Hydroxyprostaglandin dehydrogenase (15-PGDH) catalyzes the dehydrogenation of the C15 hydroxyl to produce the corresponding ketone, 15-oxo-LXA₄. Leukotriene B₄ 12-hydroxydehydrogenase (PGR/LTB4DH) may catalyze the reduction of the C13-C14 double bond of LXA₄ or 15-oxo-LXA₄ to give 13,14-dihydro-LXA₄ or 13,14-dihydro-15-oxo-LXA₄ respectively. Lipoxins are also subject to omega oxidation at C20 in certain cell types such as human neutrophils.

There is a continued need for the development of lipoxin analogs (e.g., lipoxin analogs with improved potency, stability, bioavailability, or other pharmacodynamics or pharmacokinetic properties) for the treatment of disease.

### SUMMARY OF THE INVENTION

The invention relates to (S)-6-((1R,2E,4E,8E,10S)-11-(4-fluorophenoxy)-1,10-dihydroxyundeca-2,4,8-trien-6-yn-1-yl)-1,4-dioxan-2-one (compound (1)), which is a lactone-containing analog of lipoxin A₄ (LXA₄). In particular, the invention features pharmaceutical compositions including compound (1) and the use of compound (1) for the treatment of a disorder (e.g., a fibrotic disorder or an inflammatory disorder or an autoimmune disorder) in a subject in need thereof. The invention is based, at least in part, on the realization that compound (1) has improved pharmacokinetic properties (e.g., oral bioavailability) in comparison to the corresponding free acid.

In a first aspect, the invention features a pharmaceutical composition including a therapeutically effective amount of compound (1) and a pharmaceutically acceptable excipient, wherein compound (1) has the structure:

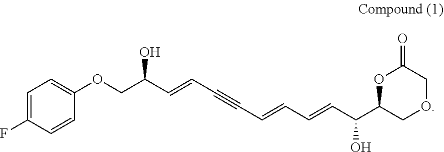 [SMILES: O=C1COC[C@@H]([C@H](O)/C=C/C=C/C#C/C=C/[C@H](O)COc2ccc(F)cc2)O1]

In another aspect, the invention features a method for treating an inflammatory disorder or an autoimmune disorder in a subject in need thereof, the method including administering to the subject a pharmaceutical composition including a therapeutically effective amount of compound (1) and a pharmaceutically acceptable excipient.

In some embodiments, the inflammatory disorder or autoimmune disorder is selected from scleroderma (e.g., systemic sclerosis, sine scleroderma, or a scleroderma-like disorder), dermatomyositis, systemic lupus erythematosus, periodontitis, an inflammatory bowel disease (IBD) (e.g., ulcerative colitis or Crohn's disease), chronic obstructive pulmonary disease (COPD), acquired immune deficiency syndrome (AIDS), multiple sclerosis, rheumatoid arthritis, psoriasis, diabetes (e.g., type 1 diabetes), cancer, asthma, atopic dermatitis, an autoimmune thyroid disorder (e.g., Graves' disease or Hashimoto's disease), stroke, ischemia, amyotrophic lateral sclerosis (ALS), chronic traumatic encephalopathy (CTE), chronic inflammatory demyelinating polyneuropathy, hepatitis, autoimmune inner ear disease, uveitis, iritis, peritonitis and a neurodegenerative disease (e.g., Alzheimer's disease or Parkinson's disease).

In another aspect, the invention features a method for treating a fibrotic disorder in a subject in need thereof, the method including administering to the subject a pharmaceutical composition including a therapeutically effective amount of compound (1) and a pharmaceutically acceptable excipient.

In some embodiments, the fibrotic disorder is selected from cystic fibrosis, scleroderma (e.g., systemic sclerosis, sine scleroderma, or a scleroderma-like disorder), liver cirrhosis, interstitial pulmonary fibrosis, idiopathic pulmonary fibrosis, Dupuytren's contracture, keloids, chronic kidney disease, chronic graft rejection, scarring or wound healing abnormalities, post-operative adhesions, reactive fibrosis, polymyositis, ANCA vasculitis, Behçet's disease, anti-phospholipid syndrome, relapsing polychondritis, familial Mediterranean fever, giant cell arteritis, Graves' ophthalmopathy, discoid lupus, pemphigus, bullous pemphigoid, hydradenitis suppuritiva, sarcoidosis, bronchiolitis obliterans, interstitial lung disease, primary sclerosing cholangitis, primary biliary cirrhosis, arterial stiffness, mediastinal fibrosis, Peyronie's disease, nephrogenic systemic fibrosis, adhesive capsulitis, dermal fibrosis, pulmonary fibrosis (e.g., radiation-induced pulmonary fibrosis, or progressive massive fibrosis), liver fibrosis, kidney fibrosis, brain fibrosis (e.g., glial scar), and heart fibrosis (e.g., atrial fibrosis, or endomyocardial fibrosis).

In some embodiments of any of the aspects described herein, the pharmaceutical composition is administered parenterally, orally, topically, nasally, rectally, buccally, by ophthalmic administration, or by inhalation. In some embodiments, the parenteral administration is intraperitoneal, subcutaneous, intracutaneous, intravenous, intramuscular, intraarticular, intraarterial, intrasynovial, intrasternal, intrathecal, intralesional, intracranial, or intracardiac.

In some embodiments of any of the aspects described herein, the pharmaceutical composition is in the form of a tablet, a capsule, a solution, an ointment, a cream, a suspension, a lotion, a powder, a paste, a gel, a spray, an aerosol, a emulsion, a dispersion, or an oil.

In some embodiments of any of the aspects described herein, the pharmaceutical composition is formulated for sustained release. In some embodiments, the pharmaceutical composition includes an enteric coating (e.g., a formulation for oral administration, such as a capsule or a tablet, including an enteric coating).

In some embodiments of any of the aspects described herein, wherein the composition is administered once daily, twice daily, or three times daily. In some embodiments, the composition is administered once a week, twice a week, once a month, or twice a month.

In some embodiments of any of the aspects described herein, the pharmaceutical composition is administered in combination with an additional therapeutic agent (e.g., an anti-inflammatory agent). In some embodiments, the additional therapeutic agent is an anti-inflammatory agent selected from a corticosteroid, a non-steroidal anti-inflammatory drug (NSAID) (e.g., acetylsalicylic acid, celecoxib, rofecoxib, valdecoxib, diclofenac, diflunisal, etodolac, ibuprofen, flurbiprofen, indomethacin, ketoprofen, ketorolac, nabumetone, naproxen, oxaprozin, piroxicam, sulindac, tolmetin, meclofenamate, mefenamic acid, or meloxicam), a biologic (e.g., inflixamab, adelimumab, etanercept, CDP-870, rituximab, or atlizumab), a small molecule immunomodulatory (e.g., VX 702, SCID 469, doramapimod, RO 30201195, SCID 323, DPC 333, pranalcasan, mycophenolate, or merimepodib), or a non-steroidal immunophilin-dependent immunosuppressant (e.g., cyclosporine, tacrolimus, pimecrolimus, or ISAtx247). Exemplary corticosteroids include algestone, 6-alpha-fluoroprednisolone, 6-alphamethylprednisolone, 6-alpha-methylprednisolone 21-acetate, 6-alpha-methylprednisolone 21-hemisuccinate sodium salt, 6-alpha,9-alpha-difluoroprednisolone 21-acetate 17-butyrate, amcinafal, beclomethasone, beclomethasone dipropionate, beclomethasone dipropionate monohydrate, 6-beta-hydroxycortisol, betamethasone, betamethasone-17-valerate, budesonide, clobetasol, clobetasol propionate, clobetasone, clocortolone, clocortolone pivalate, cortisone, cortisone acetate, cortodoxone, deflazacort, 21-deoxycortisol, deprodone, descinolone, desonide, desoximethasone, dexamethasone, dexamethasone-21-acetate, dichlorisone, diflorasone, diflorasone diacetate, diflucortolone, doxibetasol, fludrocortisone, flumethasone, flumethasone pivalate, flumoxonide, flunisolide, fluocinonide, fluocinolone acetonide, 9-fluorocortisone, fluorohydroxyandrostenedione, fluorometholone, fluorometholone acetate, fluoxymesterone, fluprednidene, fluprednisolone, flurandrenolide, formocortal, halcinonide, halometasone, halopredone, hyrcanoside, hydrocortisone, hydrocortisone acetate, hydrocortisone butyrate, hydrocortisone cypionate, hydrocortisone sodium phosphate, hydrocortisone sodium succinate, hydrocortisone probutate, hydrocortisone valerate, 6-hydroxydexamethasone, isoflupredone, isoflupredone acetate, isoprednidene, meclorisone, methylprednisolone, methylprednisolone acetate, methylprednisolone sodium succinate, paramethasone, paramethasone acetate, prednisolone, prednisolone acetate, prednisolone metasulphobenzoate, prednisolone sodium phosphate, prednisolone tebutate, prednisolone-21-hemisuccinate free acid, prednisolone-21-acetate, prednisolone-21-(beta-D-glucuronide), prednisone, prednylidene, procinonide, tralonide, triamcinolone, triamcinolone acetonide, triamcinolone acetonide 21-palmitate, triamcinolone diacetate, triamcinolone hexacetonide, and wortmannin. Desirably, the corticosteroid is fludrocortisone and prednisolone. In preferred embodiments, the corticosteroid is either fludrocortisone or prednisolone.

In some embodiments of any of the aspects described herein, the pharmaceutical composition is administered to a subject in need thereof. In some embodiments the subject is a human or an animal, such as a cat, dog, pig, rat, mouse, fish, horse, chicken, or cow. In preferred embodiments, the subject is a human.

### Definitions

To facilitate the understanding of this invention, a number of terms are defined below. Terms defined herein have meanings as commonly understood by a person of ordinary skill in the areas relevant to the invention. Terms such as “a”, “an,” and “the” are not intended to refer to only a singular entity, but include the general class of which a specific example may be used for illustration. The terminology herein is used to describe specific embodiments of the invention, but their usage does not limit the invention, except as outlined in the claims.

As used herein, the term “about” refers to a value that is within 10% above or below the value being described.

As used herein, any values provided in a range of values include both the upper and lower bounds, and any values contained within the upper and lower bounds.

As used herein, the term “treat” or “treatment” includes administration of a compound to a subject, e.g., by any route, e.g., orally, topically, by inhalation, by ex-vivo contact with one or more cells of the subject. The compound can be administered alone or in combination with one or more additional compounds. Treatments may be sequential, with the present compound being administered before or after the administration of other agents. Alternatively, compounds may be administered concurrently. The subject, e.g., a patient, can be one having a disorder (e.g., a disease or condition described herein), a symptom of a disorder, or a predisposition toward a disorder. Treatment is not limited to curing or complete healing, but can result in one or more of alleviating, relieving, altering, partially remedying, ameliorating, improving or affecting the disorder, reducing one or more symptoms of the disorder or the predisposition toward the disorder. In an embodiment the treatment (at least partially) alleviates or relieves symptoms related to a fibrotic disorder. In an embodiment the treatment (at least partially) alleviates or relieves symptoms related to an inflammatory disorder or autoimmune disorder. In one embodiment, the treatment reduces at least one symptom of the disorder or delays onset of at least one symptom of the disorder. The effect is beyond what is seen in the absence of treatment.

The term “therapeutically effective amount,” as used herein, refers to an amount, e.g., pharmaceutical dose, effective in inducing a desired effect in a subject or in treating a subject having a condition or disorder described herein (e.g., a fibrotic disorder or an inflammatory disorder, such as an autoimmune disorder). It is also to be understood herein that a “therapeutically effective amount” may be interpreted as an amount giving a desired therapeutic and/or preventative effect, taken in one or more doses or in any dosage or route, and/or taken alone or in combination with other therapeutic agents.

The term “subject,” as used herein, can be a human, non-human primate, or other mammal, such as but not limited to dog, cat, horse, cow, pig, turkey, goat, monkey, chicken, rat, mouse, and sheep.

The term “pharmaceutical composition” refers to the combination of an active agent with an excipient, inert or active, making the composition especially suitable for diagnostic or therapeutic use in vivo or ex vivo. A “pharmaceutically acceptable excipient,” after being administered to or upon a subject, does not cause undesirable physiological effects. The excipient in the pharmaceutical composition must be “acceptable” also in the sense that it is compatible with the active ingredient. The excipient may also be capable of stabilizing the active ingredient. One or more solubilizing agents can be utilized as pharmaceutical excipients for delivery of an active compound. Examples of pharmaceutically acceptable excipients include, but are not limited to, biocompatible vehicles, adjuvants, additives, and diluents to achieve a composition usable as a dosage form. Examples of other excipients include colloidal silicon oxide, magnesium stearate, cellulose, sodium lauryl sulfate, starch, glucose, lactose, sucrose, gelatin, sodium stearate, glycerol monostearate, talc, sodium chloride, glycerol, propylene glycol, water, and ethanol. Excipients can be liquids, such as water, or oils, including those of petroleum, animal, vegetable or synthetic origin, such as peanut oil, soybean oil, mineral oil, and sesame oil. Excipients can be saline, gum acacia, gelatin, starch paste, talc, keratin, colloidal silica, and urea. In addition, auxiliary, stabilizing, thickening, lubricating and coloring agents can be used. The present compositions, if desired, can also contain minor amounts of wetting or emulsifying agents, or pH buffering agents.

As used herein, the term “reduces the occurrence of adverse effects” refers to an average reduction in any adverse event experienced among human subjects when a subject or subjects are treated with a compound or a pharmaceutical composition of the invention (e.g., compound (1)) compared to a subject or subjects treated with an equivalent dose and method of administration of another therapeutic, such as another lipoxin or lipoxin analog. The adverse effects reduced using the methods of the invention can include cutaneous symptoms (e.g., diffuse edema of hands and feet, progressive skin tightening, sclerodactyly, calcinosis, telangiectasias, digital ulcers and pits, contractures, hyperpigmentation, hypopigmentation, salt and pepper skin, characteristic facies), vascular symptoms (e.g., Raynaud's phenomenon, nailfold capillary changes, digital ischemia and ulcers, vasculitic leg ulcers), pulmonary symptoms (e.g., interstitial lung disease, including alveolitis and interstitial fibrosis, pulmonary hypertension, recurrent aspiration, pneumonitis caused by esophageal reflux and dysmotility, chest wall restriction, decreased thoracic compliance, respiratory muscle weakness), cardiac symptoms (e.g., cardiomyopathy, systolic and diastolic dysfunction, congestive heart failure, conduction defects, septal infarction pattern, ventricular conduction abnormalities, arrhythmias, heart blocks, pericarditis or pericardial effusion, impending renal crisis), renal symptoms (e.g., scleroderma renal crisis, hypertension, renal failure MAHA), musculoskeletal and rheumatologic symptoms (e.g., arthralgia, tendon friction rubs, inflammatory arthritis, erosive arthropathy myopathy, myositis), gastrointestinal symptoms (e.g., gastroesophageal reflux, esophageal dysmotility, aperistaltic esophagus, esophageal stricture, adenocarcinoma arising in Barrett's esophagus, watermelon stomach, gastric antral vascular ectasias, iron-deficiency anemia, decreased peristalsis throughout the GI tract, bloating, early satiety, stasis, pseudo-obstruction, bacterial overgrowth and malabsorptive diarrhea, alternating diarrhea and constipation, megacolon, colonic wide-mouth diverticuli, pneumatosis cystoides intestinalis, primary biliary cirrhosis, anal incontinence), and endocrine symptoms (e.g., hypothyroidism), neurologic symptoms (carpal tunnel syndrome, trigeminal neuralgia). The reduction in adverse events may be a reduction of 10%, 20%, 30%, 40%, 50%, 60%, 70%, 80%, 90%, 95% or more in the occurrence or severity of an adverse events as compared to the absence of treatment or as compared to treatment with an alternate therapy.

As used herein, the a “low dosage” or “low concentration” is meant at least 5% less (e.g., at least 10%, 20%, 50%, 80%, 90%, or even 95%) than the lowest standard recommended dosage or lowest standard recommended concentration of a particular compound formulated for a given route of administration for treatment of any human disease or condition. For example, a low dosage of an anti-inflammatory, anti-microbial, or anti-viral compound formulated for oral administration will differ from a low dosage of an anti-inflammatory, anti-microbial, or anti-viral compound formulated for intravenous administration.

### DESCRIPTION OF THE DRAWINGS

FIG. 1 is a series images depicting the chemical structures of compounds (1)-(4).

FIG. 2 is a set of graphs showing that compound (1) activates human GPR32 (RvD1 Receptor). The following compounds were tested for their ability to activate the RvD1 receptor: Compound (1) (lactone), compound (2) (free acid), compound (3) (Na⁺ salt), compound (4) batch 1 (K⁺ salt), compound (4) batch 2 (K⁺ salt), resolvin D1 (RvD1), and lipoxin Aa (LXA₄) positive control. Ligand receptor interactions were monitored using the Beta-Arrestin PathHunter eXpress System™ (DiscoverX, Fremont, Calif.).

FIG. 3 is a set of graphs showing that compound (1) activates human ALX/FPR2 (LXA₄ Receptor). The following compounds were tested for their ability to activate the LXA₄ receptor: Compound (1) (lactone), compound (2) (free acid), compound (3) (Na⁺ salt), compound (4) batch 1 (K⁺ salt), compound (4) batch 2 (K⁺ salt), resolvin D1 (RvD1), and lipoxin Aa (LXA₄) positive control. Ligand receptor interactions were monitored using the Beta-Arrestin PathHunter eXpress System™ (DiscoverX, Fremont, Calif.).

FIG. 4 is a set of graphs showing that compound (1) binds to Cannabinoid Receptor 1 (CB₁) and Cannabinoid Receptor 2 (CB₂) as determined by radioligand binding assay.

FIG. 5 is a set of graphs showing that compound (2) does not bind to Cannabinoid Receptor 1 (CB₁) and Cannabinoid Receptor 2 (CB₂) as determined by radioligand binding assay.

FIG. 6 is a set of graphs and corresponding microscopy images showing that compound (1) enhances human macrophage phagocytosis of live E. coli. The following compounds were tested for their ability to enhances human macrophage phagocytosis of live E. coli: Compound (1) (lactone), compound (2) (free acid), compound (3) (Na⁺ salt), compound (4) batch 1 (K⁺ salt), compound (4) batch 2 (K⁺ salt), resolvin D1 (RvD1), lipoxin Aa (LXA₄) positive control, and vehicle. Results are recordings of mean fluorescence intensity (MFI) from one representative donor. Inset left: % increase in phagocytosis above vehicle (E. coli alone) by RvD1 and LXA₄ analogs at 100 min. Results are mean±SEM from three separate experiments. In each experiment, 4 fields (40×) per condition (per well) were recorded. Inset right: Representative fluorescent images; scale bar: 20 μm.

FIG. 7 is a set of graphs showing that compound (1) enhances human macrophage phagocytosis of live E. coli (dose response). The following compounds were tested for their ability to enhance human macrophage phagocytosis of live E. coli: Compound (1) (lactone), compound (2) (free acid), compound (3) (Na⁺ salt), compound (4) batch 1 (K⁺ salt), compound (4) batch 2 (K⁺ salt), resolvin D1 (RvD1), and lipoxin Aa (LXA₄) positive control. Results are expressed as percent increase above vehicle (E. coli alone); mean from 4 separate experiments.

FIG. 8 is a graph showing that compound (1) accelerates PMN clearance in vivo in a mouse model of E. coli initiated peritonitis.

FIG. 9 is a set of graphs showing that compound (1) accelerates PMN clearance in vivo in a mouse model of E. coli initiated peritonitis.

FIG. 10 is a graph showing absolute total nucleated cells in peritoneal lavage. Supernatant in a mouse peritonitis model 3 hours post-administration of 0.1 μg/mouse or 10 μg/mouse of compound (1).

FIG. 11 is a graph showing the effect of compound (1) in an experimental allergic encephalomyelitis (EAE) model.

FIG. 12 is a graph showing the plasma concentration of compound (2) following administration of either compound (1) or compound (2) in rat orally dosed with 10 mg/kg.

FIG. 13 is a graph showing the plasma concentration of compound (2) following oral administration of either compound (1) or compound (2) in rat intravenously (IV) dosed with 0.3 mg/kg.

### DETAILED DESCRIPTION OF THE INVENTION

In general, the invention relates to (S)-6-((1R,2E,4E,8E,10S)-11-(4-fluorophenoxy)-1,10-dihydroxyundeca-2,4,8-trien-6-yn-1-yl)-1,4-dioxan-2-one (compound (1)), which is a lactone-containing analog of lipoxin Aa (LXA₄). In particular, the invention features pharmaceutical compositions including compound (1) and the use of compound (1) for the treatment of a disorder (e.g., a fibrotic disorder or an inflammatory disorder, such as an autoimmune disorder) in a subject in need thereof.

Compounds

The disclosure provides compositions and methods relating to (S)-6-((1R,2E,4E,8E,10S)-11-(4-fluorophenoxy)-1,10-dihydroxyundeca-2,4,8-trien-6-yn-1-yl)-1,4-dioxan-2-one (compound (1)):

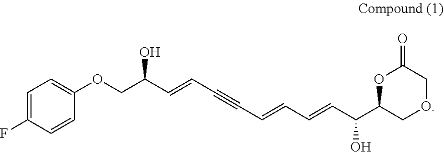 [SMILES: O=C1COC[C@@H]([C@H](O)/C=C/C=C/C#C/C=C/[C@H](O)COc2ccc(F)cc2)O1]

The method features pharmaceutical compositions including compound (1) and a pharmaceutically acceptable excipient. The invention also features methods for treating, preventing, or ameliorating a disease, condition, or disorder (e.g., a fibrotic disease or an inflammatory disorder, such as an autoimmune disorder) in a subject in need thereof by administering to the subject a pharmaceutical composition including a therapeutically effective amount of compound (1) (e.g., a pharmaceutical composition including compound (1) and a pharmaceutically acceptable excipient).

Compound (1) is a lactone analog of a 3-oxa-15-epi-LXA₄ compound: 2-((13-(4-fluorophenoxy)-2,3,12-trihydroxytrideca-4,6,10-trien-8-yn-1-yl)oxy)acetate (also known as ZK-818994 (free acid, CAS 524714-13-4), ZK-821192 or ZK-994 (Na⁺ salt, CAS 686773-12-6), ZK-994 (Na⁺ salt, CAS 1030359-59-1), ZK-355277 (K⁺ salt)). Applicants have surprisingly discovered that compound (1) can exhibit improved therapeutic properties (e.g., improved pharmacokinetic properties and/or improved potency) over the corresponding free acid, Na⁺ salt, or K⁺ salt. Compounds with improved pharmacokinetic properties or pharmacodynamic properties may be administered, for example, at lower doses, enabling an improved safety and/or efficacy profile.

A compound of the invention (e.g., compound (1)) may display an increased safety and/or efficacy profile in the treatment of a disease or condition (e.g., a fibrotic disorder or an inflammatory disorder, such as an autoimmune disorder), as compared to other therapies for the treatment of an inflammatory disease (e.g., an autoimmune disorder) or a fibrotic disease, such as other lipoxins or lipoxin analogs. In some embodiments, administration of a compound of the invention (e.g., compound (1)) to a subject (e.g., a subject having a disease or condition described herein) results in a decrease in treatment-associated adverse events relative to treatment with another therapy for the treatment of an inflammatory disease (e.g., an autoimmune disorder) or a fibrotic disease, such as other lipoxins or lipoxin analogs. The reduction in adverse events may be a reduction of 10%, 20%, 30%, 40%, 50%, 60%, 70%, 80%, 90%, 95% or more in the occurrence or severity of an adverse events (e.g., compared to a subject or subjects treated with an equivalent dose and method of administration of another lipoxin or lipoxin analog).

Pharmaceutical Compositions

Compounds of the invention (e.g., compound (1)) prepared by any of the methods described herein may be formulated as a pharmaceutical composition for the treatment of disease. As described above, the pharmaceutical compositions of the invention additionally include a pharmaceutically acceptable excipient, which, as used herein, includes any and all solvents, diluents, or other liquid vehicle, dispersion or suspension aids, surface active agents, isotonic agents, thickening or emulsifying agents, preservatives, solid binders, and lubricants, as suited to the particular dosage form desired. Remington (The Science and Practice of Pharmacy, 22nd Edition, A. R. Gennaro, Lippincott, Williams & Wilkins, Baltimore, Md., 2012) discloses various excipients used in formulating pharmaceutical compositions and known techniques for the preparation thereof. Except insofar as any conventional excipient medium is incompatible with the compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable excipients include, but are not limited to, sugars such as lactose, glucose and sucrose; sugar alcohols such as mannitol and sorbitol; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; natural and synthetic phospholipids, such as soybean and egg yolk phosphatides, lecithin, hydrogenated soy lecithin, dimyristoyl lecithin, dipalmitoyl lecithin, distearoyl lecithin, dioleoyl lecithin, hydroxylated lecithin, lysophosphatidylcholine, cardiolipin, sphingomyelin, phosphatidylcholine, phosphatidyl ethanolamine, diastearoyl phosphatidylethanolamine (DSPE) and its pegylated esters, such as DSPE-PEG750 and, DSPE-PEG2000, phosphatidic acid, phosphatidyl glycerol and phosphatidyl serine. Commercial grades of lecithin which are preferred include those which are available under the trade name Phosal® or Phospholipon® and include Phosal 53 MCT, Phosal 50 PG, Phosal 75 SA, Phospholipon 90H, Phospholipon 90G and Phospholipon 90 NG; soy-phosphatidylcholine (SoyPC) and DSPE-PEG2000 are particularly preferred; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The above-described compositions (e.g., compositions including compound (1)), in any of the forms described herein, can be used for treating fibrotic disorder, inflammatory disorder (e.g., an autoimmune disorder), or any other disease or condition described herein. An effective amount refers to the amount of an active compound/agent that is required to confer a therapeutic effect on a treated subject. Effective doses will vary, as recognized by those skilled in the art, depending on the types of diseases treated, route of administration, excipient usage, and the possibility of co-usage with other therapeutic treatment.

A pharmaceutical composition of this invention can be administered parenterally, orally, nasally, rectally, topically, buccally, by ophthalmic administration, or by inhalation. The term “parenteral” as used herein refers to subcutaneous, intracutaneous, intravenous, intramuscular, intraarticular, intraarterial, intrasynovial, intrasternal, intrathecal, intralesional, intracranial, intracardiac, or intraperitoneal injection, as well as any suitable infusion technique.

A sterile injectable composition can be a solution or suspension in a non-toxic parenterally acceptable diluent or solvent. Such solutions include, but are not limited to, 1,3-butanediol, mannitol (e.g., aqueous mannitol solution), water, Ringer's solution, and isotonic sodium chloride solution. In addition, fixed oils are conventionally employed as a solvent or suspending medium (e.g., synthetic mono- or diglycerides). Fatty acids, such as, but not limited to, oleic acid and its glyceride derivatives, are useful in the preparation of injectables, as are natural pharmaceutically acceptable oils, such as, but not limited to, olive oil or castor oil, or polyoxyethylated versions thereof. These oil solutions or suspensions also can contain a long chain alcohol diluent or dispersant such as, but not limited to, carboxymethyl cellulose, or similar dispersing agents. Other commonly used surfactants, such as, but not limited to, Tweens or Spans or other similar emulsifying agents or bioavailability enhancers, which are commonly used in the manufacture of pharmaceutically acceptable solid, liquid, or other dosage forms also can be used for the purpose of formulation.

A composition for oral administration can be any orally acceptable dosage form including capsules, tablets (e.g. a pressed tablet), emulsions and aqueous suspensions, dispersions, and solutions. In the case of tablets, commonly used excipients include, but are not limited to, lactose and corn starch. Lubricating agents, such as, but not limited to, magnesium stearate, also are typically added. For oral administration in a capsule form, useful diluents include, but are not limited to, lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

Pharmaceutical compositions for topical administration according to the described invention can be formulated as solutions, ointments, creams, suspensions, lotions, powders, pastes, gels, sprays, aerosols, or oils. Alternatively, topical formulations can be in the form of patches or dressings impregnated with active ingredient(s), which can optionally include one or more excipients or diluents. In some preferred embodiments, the topical formulations include a material that would enhance absorption or penetration of the active agent(s) through the skin or other affected areas.

A topical composition contains a safe and effective amount of a dermatologically acceptable excipient suitable for application to the skin. A “cosmetically acceptable” or “dermatologically-acceptable” composition or component refers to a composition or component that is suitable for use in contact with human skin without undue toxicity, incompatibility, instability, or allergic response. The excipient enables an active agent and optional component to be delivered to the skin at an appropriate concentration(s). The excipient thus can act as a diluent, dispersant, solvent, or the like to ensure that the active materials are applied to and distributed evenly over the selected target at an appropriate concentration. The excipient can be solid, semi-solid, or liquid. The excipient can be in the form of a lotion, a cream, or a gel, in particular one that has a sufficient thickness or yield point to prevent the active materials from sedimenting. The excipient can be inert or possess dermatological benefits. It also should be physically and chemically compatible with the active components described herein, and should not unduly impair stability, efficacy, or other use benefits associated with the composition.

Pharmaceutical Dosage Forms

Various dosage forms of a compound of the invention (e.g., compound (1)) produced by any of the methods described herein can be used for preventing and/or treating a condition (e.g., an inflammatory disorder, such as an autoimmune disorder, or a fibrotic disorder). In some embodiments, the dosage form is an oral dosage form such as a pressed tablet, hard or soft gel capsule, enteric coated tablet, osmotic release capsule, or unique combination of excipients.

In further embodiments, the dosage form includes an additional agent or is provided together with a second dosage form, which includes the additional agent. Exemplary additional agents include an analgesic agent such as an NSAID or opiate, an anti-inflammatory agent or a natural agent such as a triglyceride containing unsaturated fatty acid, or isolated pure fatty acids such as eicosapentaenoic acid (EPA), dihomogamma linolenic acid (DGLA), docosahexaenoic acid (DHA) and others. In additional embodiments, the dosage form includes a capsule wherein the capsule contains a mixture of materials to provide a desired sustained release formulation.

The dosage forms can include a tablet coated with a semipermeable coating. In certain embodiments, the tablet includes two layers, a layer containing a compound of the invention (e.g. compound (1)) and a second layer referred to as a “push” layer. The semi-permeable coating is used to allow a fluid (e.g., water) to enter the tablet and erode a layer or layers. In certain embodiments, this sustained release dosage form further includes a laser hole drilled in the center of the coated tablet. The layer containing the compound of the invention may include a compound of the invention (e.g., compound (1)), a disintegrant, a viscosity enhancing agent, a binding agent, and an osmotic agent. The push layer may include a disintegrant, a binding agent, an osmotic agent, and a viscosity enhancing agent.

The present compositions may be formulated for sustained release (e.g., over a 2 hour period, over a 6 hour period, over a 12 hour period, over a 24 hour period, over a 48 hour period, over 1 week, over 2 weeks, over 1 month, over 6 months or more).

In further embodiments, the dosage form includes a tablet including a biocompatible matrix and a compound of the invention (e.g., compound (1)). The sustained release dosage form may also include a hard-shell capsule containing bio-polymer microspheres that contains the therapeutically active agent. Formulation including a biocompatible matrix and/or bio-polymer microspheres each contain pores for drug release and delivery. These pores are formed by mixing the biocompatible matrix of bio-polymer microsphere with a pore forming agent. Each biocompatible matrix or bio-polymer microsphere is made up of a biocompatible polymer or mixture of biocompatible polymers. The matrix and microspheres can be formed by dissolving the biocompatible polymer and active agent (e.g., compound (1)) in a solvent and adding a pore-forming agent (e.g., a volatile salt). Evaporation of the solvent and pore forming agent provides a matrix or microsphere containing the active compound. In additional embodiments, the sustained release dosage form includes a tablet, wherein the tablet contains a compound of the invention (e.g., compound (1)) and one or more polymers and wherein the tablet can be prepared by compressing the compound (e.g., e.g., compound (1)) and one or more polymers. In some embodiments, the one or more polymers may include a hygroscopic polymer formulated with the compound (e.g., compound (1)). Upon exposure to moisture, the tablet dissolves and swells. This swelling allows the sustained release dosage form to remain in the upper GI tract. The swelling rate of the polymer mixture can be varied using different grades of polyethylene oxide.

In other embodiments, the sustained release dosage form includes a capsule further including particle cores coated with a suspension of active agent and a binding agent which is subsequently coated with a polymer. The polymer may be a rate-controlling polymer. In general, the delivery rate of the rate-controlling polymer is determined by the rate at which the active agent is dissolved.

In some embodiments, the pharmaceutical composition includes an enteric coating (e.g., a formulation for oral administration, such as a capsule or a tablet, including an enteric coating). An enteric coating is any barrier (e.g., polymer barrier) applied on oral medication that prevents its dissolution or disintegration in the gastric environment. This helps by either protecting drugs from the acidity of the stomach, the stomach from the detrimental effects of the drug, or to release the drug after the stomach (usually in the upper tract of the intestine). In some embodiments, the enteric coating includes one or more of a methyl acrylate-methacrylic acid copolymer, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymer, shellac, cellulose acetate trimellitate, sodium alginate, zein, enteric coating aqueous solution, ethylcellulose, medium chain triglycerides, oleic acid, and sodium stearic acid.

In some embodiments, the tablet or capsule comprises an inner component surrounding the composition and an outer component, the latter serving as an envelope over the former. The two components can be separated by an enteric coating layer that may resist disintegration in the stomach and permits the inner component to pass intact into the duodenum or to be delayed in release.

In some embodiments, one or more of the therapeutic agents may be formulated with a pharmaceutically acceptable carrier, vehicle or adjuvant. The term “pharmaceutically acceptable carrier, vehicle, or adjuvant” refers to a carrier, vehicle or adjuvant that may be administered to a subject, together with the present compounds, and which does not destroy the pharmacological activity thereof and is nontoxic when administered in doses sufficient to deliver a therapeutic amount of the compound.

Pharmaceutically acceptable carriers, adjuvants and vehicles that may be used in the dosage forms of this invention include, but are not limited to, ion exchangers, alumina, aluminum stearate, lecithin, self-emulsifying drug delivery systems (SEDDS) such as d-E-tocopherol polyethylene-glycol 1000 succinate; surfactants used in pharmaceutical dosage forms such as Tweens or other similar polymeric delivery matrices; serum proteins such as human serum albumin; buffer substances such as phosphates, glycine, sorbic acid, potassium sorbate, partial glyceride mixtures of saturated vegetable fatty acids, water, salts; or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxmethylcellulose, polyacrylates, waxes, polyethylene-polyoxypropylene-block polymers, polyethylene glycol and wool fat. Cyclodextrins such as alpha, beta and gamma-cyclodextrin, or chemically modified derivatives such as hydroxyalkylcyclodextrins, including 2- and 3-hydroxypropyl-beta cyclodextrins, or other solubilized derivatives may also be advantageously used to enhance delivery of compounds of the formulae described herein that can be used in the methods of the invention for preventing and/or treating fibrotic conditions or inflammatory conditions (e.g., autoimmune conditions). In certain embodiments, the pharmaceutically acceptable carrier, adjuvant, or vehicle includes a polymer or wax, such as a polymer of wax in a spray dried dispersion or hot melt extrusion formulation. In certain embodiments, unit dosage formulations are compounded for immediate release, though unit dosage formulations compounded for delayed or prolonged release of one or both agents are also disclosed.

In some embodiments, one or more therapeutic agents (e.g., compound (1)) may be formulated in a single unit dose such that the agents are released from the dosage at different times. In another embodiment, for example, where one or more of the therapeutic agents is administered once or twice per day, the agent is formulated to provide extended release. For example, the agent is formulated with an enteric coating. In an alternative embodiment, the agent is formulated using a biphasic controlled release delivery system, thereby providing prolonged gastric residence. For example, in some embodiments, the delivery system includes (1) an inner solid particulate phase formed of substantially uniform granules containing a pharmaceutical having a high water solubility, and one or more hydrophilic polymers, one or more hydrophobic polymers and/or one or more hydrophobic materials such as one or more waxes, fatty alcohols and/or fatty acid esters, and (2) an outer solid continuous phase in which the above granules of inner solid particulate phase are embedded and dispersed throughout, the outer solid continuous phase including one or more hydrophilic polymers, one or more hydrophobic polymers and/or one or more hydrophobic materials such as one or more waxes, fatty alcohols and/or fatty acid esters, which may be compressed into tablets or filled into capsules. In some embodiments, the agent is incorporated into polymeric matrices comprised of hydrophilic polymers that swell upon imbibition of water to a size that is large enough to promote retention of the dosage form in the stomach during the fed mode.

One or more therapeutic agents (e.g., compound (1)) may be formulated as a combination of fast-acting and controlled release forms. For example, one or more therapeutic agents (e.g., compound (1)) may be formulated with a single release property. For example, it is not present in a modified release form, e.g., a controlled release form.

The present compositions may be taken just prior to or with each of three meals, each of two major meals, or one meal. In other embodiments, a composition disclosed herein can be administered one or more times daily (e.g., once daily, twice daily, or three times daily) and need not be administered just before or with a meal.

The present compounds or compositions may be administered orally, for example as a component in a dosage form. The dosage forms may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form.

The dosage forms of this invention may be orally administered in any orally acceptable dosage form including, but not limited to, capsules, tablets, emulsions and aqueous suspensions, dispersions and solutions. In the case of tablets for oral use, carriers that are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase and may be combined with emulsifying and/or suspending agents. If desired, certain sweetening and/or flavoring and/or coloring agents may be added.

Non-limiting examples of capsules include but are not limited to gelatin capsules, HPMC, hard shell, soft shell, or any other suitable capsule for holding a sustained release mixture. The solvents used in the above sustained release dosage forms include, but are not limited to ethyl acetate, triacetin, dimethyl sulfoxide (DIV1S0), propylene carbonate, N-methylpyrrolidone (NMP), ethyl alcohol, benzyl alcohol, glycofurol, alpha-tocopherol, Miglyol 810, isopropyl alcohol, diethyl phthalate, polyethylene glycol 400 (PEG 400), triethyl citrate, and benzyl benzoate.

The viscosity modifiers that may be used in the above pharmaceutical compositions include, but are not limited to, caprylic/capric triglyceride (Miglyol 810), isopropyl myristate (IPM), ethyl oleate, triethyl citrate, dimethyl phthalate, benzyl benzoate and various grades of polyethylene oxide. The high viscosity liquid carriers used in the above sustained release dosage forms include, but are not limited to sucrose acetate isobutyrate (SA1B) and cellulose acetate butyrate (CAB) 381-20.

Non-limiting examples of materials that make up preferred semi-permeable layers include, but are not limited to cellulosic polymers such as cellulose acetate, cellulose acylate, cellulose diacylate, cellulose triacylate, cellulose diacetate, cellulose triacetate or any mixtures thereof; ethylene vinyl acetate copolymers, polyethylene, copolymers of ethylene, polyolefins including ethylene oxide copolymers (e.g., Engage®—Dupont Dow Elastomers), polyamides, cellulosic materials, polyurethanes, polyether blocked amides, and copolymers (e.g., PEBAX®, cellulosic acetate butyrate and polyvinyl acetate). Non-limiting examples of disintegrants that may be employed in the above sustained release dosage forms include but are not limited to croscarmellose sodium, crospovidone, sodium alginate or similar excipients.

Non-limiting examples of binding agents that may be employed in the above dosage forms include, but are not limited to, hydroxyalkylcellulose, a hydroxyalkylalkylcellulose, or a polyvinylpyrrolidone.

Non-limiting examples of osmotic agents that may be employed in the above dosage forms include, but are not limited to, sorbitol, mannitol, sodium chloride, or other salts. Non-limiting examples of biocompatible polymers employed in the above sustained release dosage forms include but are not limited to poly(hydroxy acids), polyanhydrides, polyorthoesters, polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polysiloxanes, poly(vinyl alcohols), poly(vinyl acetate), polystyrene, polyurethanes and co-polymers thereof, synthetic celluloses, polyacrylic acids, poly(butyric acid), poly(valeric acid), and poly(lactide-co-caprolactone), ethylene vinyl acetate, copolymers and blends thereof.

Non-limiting examples of hygroscopic polymers that may be employed in the above dosage forms include but are not limited to polyethylene oxide (e.g., Polyox® with MWs from 4,000,000 to 10,000,000), cellulose hydroxymethyl cellulose, hydroxyethyl-cellulose, crosslinked polyacrylic acids and xanthan gum.

Non-limiting examples of rate-controlling polymers may be employed in the above dosage forms include but are not limited to polymeric acrylate, methacrylate lacquer or mixtures thereof, polymeric acrylate lacquer, methacrylate lacquer, an acrylic resin including a copolymer of acrylic and methacrylic acid esters or an ammonium methacrylate lacquer with a plasticizer.

Methods of Treatment

Any of the above-described compounds or pharmaceutical compositions (e.g., a pharmaceutical including compound (1)) may be administered to a subject (e.g., a mammal, such as a human, cat, dog, horse, cow, or pig) having a disease (e.g., a fibrotic disorder or an inflammatory disorder, such as an autoimmune disorder) in order to treat, prevent, or ameliorate the disease.

Pharmaceutical compositions of the invention include a therapeutically effective amount of an active compound (e.g., compound (1)), where the therapeutically effective amount is an amount, e.g., pharmaceutical dose, effective in inducing a desired effect in a subject or in treating a subject having a condition or disorder described herein (e.g., a fibrotic disorder or an inflammatory disorder, such as an autoimmune disorder). Treatment is not limited to curing or complete healing, but can result in one or more of alleviating, relieving, altering, partially remedying, ameliorating, improving or affecting the disorder, reducing one or more symptoms of the disorder or the predisposition toward the disorder.

A therapeutically effective amount of a compound of the invention (e.g., compound (1)), such as in a dosage form of the invention, may be 0.1 mg, 0.5 mg, 1±0.2 mg, 2.0±0.5 mg, 5±1 mg, 10±2 mg, 20±5 mg, 30±5 mg, 40±5 mg, or 50±5 mg, or between 0.1-0.5 mg, 0.5-1 mg, 1-2 mg, 2-5 mg, 5-10 mg, 10-20 mg, 20-30 mg, 30-40 mg, or 40-50 mg. The dosage form, e.g., any of the dosage forms described herein, may be administered to a subject once daily, twice daily, three times daily, once every two days, once weekly, once every two weeks, once a month, or once every six months.

Therapy according to the invention may be performed alone or in conjunction with another therapy and may be provided at home, the doctor's office, a clinic, a hospital's outpatient department, or a hospital. Treatment optionally begins at a hospital so that the doctor can observe the therapy's effects closely and make any adjustments that are needed, or it may begin on an outpatient basis. The duration of the therapy depends on the type of disease or disorder being treated, the age and condition of the patient, the stage and type of the patient's disease, and how the patient responds to the treatment. Additionally, a person having a greater risk of developing an inflammatory disorder (e.g., an autoimmune disorder) or a fibrotic disorder may receive treatment to inhibit or delay the onset of symptoms.

Routes of administration for the various embodiments include, but are not limited to, parenteral, oral, topical, nasal, rectal, buccal, ophthalmic, inhalation, via an implant, or via a patch. Parenteral administration may be intraperitoneal, subcutaneous, intracutaneous, intravenous, intramuscular, intraarticular, intraarterial, intrasynovial, intrasternal, intrathecal, intralesional, intracranial, or intracardiac.

Inflammation

Any of the above-described compounds or pharmaceutical compositions (e.g., a pharmaceutical including compound (1)) may be administered to a subject having an inflammatory disorder or an autoimmune disorder in order to treat, prevent, or ameliorate the inflammatory or autoimmune disorder. Inflammatory disorder or autoimmune disorders contemplated by the present invention include, but are not limited to, scleroderma (e.g., systemic sclerosis, sine scleroderma, or a scleroderma-like disorder), dermatomyositis, systemic lupus erythematosus, periodontitis, inflammatory bowel disease (IBD) (e.g., ulcerative colitis or Crohn's disease), chronic obstructive pulmonary disease (COPD), acquired immune deficiency syndrome (AIDS), multiple sclerosis, rheumatoid arthritis, psoriasis, diabetes (e.g., type 1 diabetes), cancer, asthma, atopic dermatitis, autoimmune thyroid disorders (e.g., Graves' disease or Hashimoto's disease), stroke, ischemia, amyotrophic lateral sclerosis (ALS), chronic traumatic encephalopathy (CTE), chronic inflammatory demyelinating polyneuropathy, hepatitis, autoimmune inner ear disease, uveitis, iritis, peritonitis and a neurodegenerative diseases (e.g., Alzheimer's disease or Parkinson's disease). The treatment of inflammatory disorders or autoimmune disorders also includes the treatment of pain associated with inflammation. Administration of a compound or pharmaceutical composition described herein may reduce pain associated with inflammation (e.g., pain associated with an inflammatory or autoimmune disorder).

Inflammation can be assayed by measuring the chemotaxis and activation state of inflammatory cells. In some embodiments, inflammation can be measured by examining the production of specific inflammatory mediators such as interleukins, cytokines and eicosanoids. In some embodiments, in vivo inflammation is measured by swelling and edema of a localized tissue or migration of leukocytes. Inflammation may also be measured by organ function such as in the lung or kidneys and by the production of pro-inflammatory factors. Inflammation may also be assessed by other suitable methods. Other methods known to one skilled in the art may also be suitable methods for the assessment of inflammation and may be used to evaluate or score the response of the subject to treatment with one or more therapeutic agents of the invention (e.g., compound (1)).

Fibrotic Disorders

Any of the above-described compounds or pharmaceutical compositions (e.g., a pharmaceutical including compound (1)) may be administered to a subject having a fibrotic disorder in order to treat, prevent, or ameliorate the fibrotic disorder. Fibrotic disorders contemplated by the present disclosure include, but are not limited to, cystic fibrosis, scleroderma (e.g., systemic sclerosis, sine scleroderma, or a scleroderma-like disorder), liver cirrhosis, interstitial pulmonary fibrosis, idiopathic pulmonary fibrosis, Dupuytren's contracture, keloids, chronic kidney disease, chronic graft rejection, scarring or wound healing abnormalities, post-operative adhesions, reactive fibrosis, polymyositis, ANCA vasculitis, Behçet's disease, anti-phospholipid syndrome, relapsing polychondritis, familial Mediterranean fever, giant cell arteritis, Graves' ophthalmopathy, discoid lupus, pemphigus, bullous pemphigoid, hydradenitis suppuritiva, sarcoidosis, bronchiolitis obliterans, interstitial lung disease, primary sclerosing cholangitis, primary biliary cirrhosis, arterial stiffness, mediastinal fibrosis, Peyronie's disease, nephrogenic systemic fibrosis, adhesive capsulitis, dermal fibrosis, pulmonary fibrosis (e.g., radiation-induced pulmonary fibrosis, or progressive massive fibrosis), liver fibrosis, kidney fibrosis, brain fibrosis (e.g., glial scar), and heart fibrosis (e.g., atrial fibrosis, or endomyocardial fibrosis).

Non-limiting examples of fibrosis also include liver fibrosis, lung fibrosis (e.g., silicosis, asbestosis, and idiopathic pulmonary fibrosis), oral fibrosis, endomyocardial fibrosis, retroperitoneal fibrosis, deltoid fibrosis, kidney fibrosis (including diabetic nephropathy), cystic fibrosis, and glomerulosclerosis. Liver fibrosis, for example, occurs as a part of the wound-healing response to chronic liver injury. Fibrosis can occur as a complication of haemochromatosis, Wilson's disease, alcoholism, schistosomiasis, viral hepatitis, bile duct obstruction, exposure to toxins, and metabolic disorders. Endomyocardial fibrosis is an idiopathic disorder that is characterized by the development of restrictive cardiomyopathy. In endomyocardial fibrosis, the underlying process produces patchy fibrosis of the endocardial surface of the heart, leading to reduced compliance and, ultimately, restrictive physiology as the endomyocardial surface becomes more generally involved. Oral submucous fibrosis is a chronic, debilitating disease of the oral cavity characterized by inflammation and progressive fibrosis of the submucosal tissues (lamina propria and deeper connective tissues). The buccal mucosa is the most commonly involved site, but any part of the oral cavity can be involved, even the pharynx. Retroperitoneal fibrosis is characterized by the development of extensive fibrosis throughout the retroperitoneum, typically centered over the anterior surface of the fourth and fifth lumbar vertebrae.

A therapeutically effective amount of any of the compositions described herein (e.g. a compound (1)) may be used to treat or prevent fibrosis. Fibrosis may be assessed by suitable methods known to one of skill in the art.

Scleroderma

Scleroderma is a disease of the connective tissue characterized by fibrosis and inflammation of the skin and internal organs. Scleroderma has a spectrum of manifestations and a variety of therapeutic implications. It includes localized scleroderma, systemic sclerosis, scleroderma-like disorders, and sine scleroderma. Systemic sclerosis can be diffuse or limited. Limited systemic sclerosis is also called CREST (calcinosis, Raynaud's esophageal dysfunction, sclerodactyly, telangiectasia). Systemic sclerosis includes: scleroderma lung disease, scleroderma renal crisis, cardiac manifestations, muscular weakness including fatigue or limited CREST, gastrointestinal dysmotility and spasm, and abnormalities in the central, peripheral and autonomic nervous system.

The major symptoms or manifestations of scleroderma, and in particular of systemic sclerosis, are inappropriate excessive collagen synthesis and deposition, endothelial dysfunction, vasospasm, collapse and obliteration of vessels by fibrosis. In terms of diagnosis, an important clinical parameter may be skin thickening proximal to the metacarpophalangeal joints. Raynaud's phenomenon may be a component of scleroderma. Raynaud's may be diagnosed by color changes of the skin upon cold exposure. Ischemia and skin thickening may also be symptoms of Raynaud's disease.

A therapeutically effective amount of any of the compositions described herein (e.g. a compound (1)) may be used to treat or prevent scleroderma. Scleroderma may be assessed by suitable methods known to one of skill in the art.

Combination Therapies

A composition of the invention (e.g., a pharmaceutical composition including compound (1)) may be administered in combination with one or more additional therapeutic agents. For example, anti-inflammatory agents are useful for treating an inflammatory disorder or a fibrotic disorder in combination with compound (1). The anti-inflammatory agent may be selected from, for example, ajulemic acid (i.e. lenabasum, anabasum, JBT-101), a corticosteroid, a non-steroidal anti-inflammatory drug (NSAID) (e.g., acetylsalicylic acid, celecoxib, rofecoxib, valdecoxib, diclofenac, diflunisal, etodolac, ibuprofen, flurbiprofen, indomethacin, ketoprofen, ketorolac, nabumetone, naproxen, oxaprozin, piroxicam, sulindac, tolmetin, meclofenamate, mefenamic acid, or meloxicam), a biologic (e.g., inflixamab, adelimumab, etanercept, CDP-870, rituximab, or atlizumab), a small molecule immunomodulatory (e.g., VX 702, SCIO 469, doramapimod, RO 30201195, SCIO 323, DPC 333, pranalcasan, mycophenolate, and merimepodib), or a non-steroidal immunophilin-dependent immunosuppressant (e.g., cyclosporine, tacrolimus, pimecrolimus, or ISAtx247).

Exemplary corticosteroids include algestone, 6-alpha-fluoroprednisolone, 6-alphamethylprednisolone, 6-alpha-methylprednisolone 21-acetate, 6-alpha-methylprednisolone 21-hemisuccinate sodium salt, 6-alpha,9-alpha-difluoroprednisolone 21-acetate 17-butyrate, amcinafal, beclomethasone, beclomethasone dipropionate, beclomethasone dipropionate monohydrate, 6-beta-hydroxycortisol, betamethasone, betamethasone-17-valerate, budesonide, clobetasol, clobetasol propionate, clobetasone, clocortolone, clocortolone pivalate, cortisone, cortisone acetate, cortodoxone, deflazacort, 21-deoxycortisol, deprodone, descinolone, desonide, desoximethasone, dexamethasone, dexamethasone-21-acetate, dichlorisone, diflorasone, diflorasone diacetate, diflucortolone, doxibetasol, fludrocortisone, flumethasone, flumethasone pivalate, flumoxonide, flunisolide, fluocinonide, fluocinolone acetonide, 9-fluorocortisone, fluorohydroxyandrostenedione, fluorometholone, fluorometholone acetate, fluoxymesterone, fluprednidene, fluprednisolone, flurandrenolide, formocortal, halcinonide, halometasone, halopredone, hyrcanoside, hydrocortisone, hydrocortisone acetate, hydrocortisone butyrate, hydrocortisone cypionate, hydrocortisone sodium phosphate, hydrocortisone sodium succinate, hydrocortisone probutate, hydrocortisone valerate, 6-hydroxydexamethasone, isoflupredone, isoflupredone acetate, isoprednidene, meclorisone, methylprednisolone, methylprednisolone acetate, methylprednisolone sodium succinate, paramethasone, paramethasone acetate, prednisolone, prednisolone acetate, prednisolone metasulphobenzoate, prednisolone sodium phosphate, prednisolone tebutate, prednisolone-21-hemisuccinate free acid, prednisolone-21-acetate, prednisolone-21-(beta-D-glucuronide), prednisone, prednylidene, procinonide, tralonide, triamcinolone, triamcinolone acetonide, triamcinolone acetonide 21-palmitate, triamcinolone diacetate, triamcinolone hexacetonide, and wortmannin. Desirably, the corticosteroid is fludrocortisone and prednisolone. In preferred embodiments, the corticosteroid is either fludrocortisone or prednisolone.

Such compounds can act synergistically with compound (1) (e.g., such that the combined effect of Compound (1) and the additional therapeutic agent is greater than the sum of each agent administered alone). Additionally, coadministration with compound (1) may result in the efficacy of the anti-inflammatory compound at lower (and thus safer) dose (e.g., at least 5%, 10%, 20%, 50%, 80%, 90%, or even 95% less) than when the anti-inflammatory compound is administered alone.

In combination therapy (e.g., compound (1) with one or more additional therapeutic agents), the dosage and frequency of administration of each component of the combination can be controlled independently. For example, one compound may be administered three times per day, while the second compound may be administered once per day. Combination therapy may be given in on-and-off cycles that include rest periods so that the patients body has a chance to recover from any as yet unforeseen side effects. The compounds may also be formulated together such that one administration delivers both compounds.

Each compound of the combination may be formulated in a variety of ways that are known in the art. For example, the first and second agents may be formulated together or separately. Desirably, the first and second agents are formulated together for the simultaneous or near simultaneous administration of the agents. Such co-formulated compositions can include the two drugs together in the same pill, ointment, cream, foam, capsule, liquid, etc. It is to be understood that, when referring to the formulation of combinations of the invention, the formulation technology employed is also useful for the formulation of the individual agents of the combination, as well as other combinations of the invention. By using different formulation strategies for different agents, the pharmacokinetic profiles for each agent can be suitably matched.

Kits

The individually or separately formulated agents (e.g., compound (1) or compound (1) in combination with one or more additional therapeutic agents) can be packaged together as a kit. Nonlimiting examples include kits that contain, e.g., two pills, a pill and a powder, a suppository and a liquid in a vial, two topical creams, ointments, foams etc. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1. Synthesis of (S)-6-((1R,2E,4E,8E,10S)-11-(4-fluorophenoxy)-1,10-dihydroxyundeca-2,4,8-trien-6-yn-1-yl)-1,4-dioxan-2-one (Compound (1))

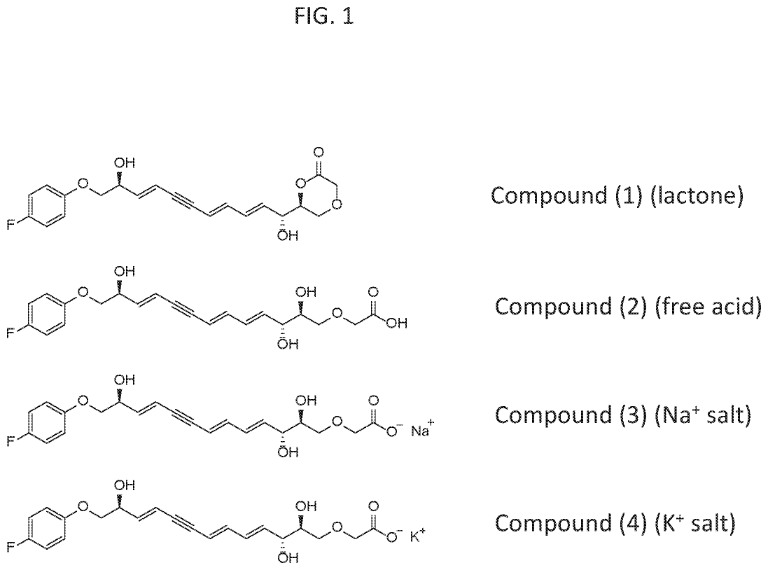

Compound (1) is the lactone analog of a 3-oxa-15-epi-LXA₄ compound: 2-(((2S,3R,4E,6E,10E,12S)-13-(4-fluorophenoxy)-2,3,12-trihydroxytrideca-4,6,10-trien-8-yn-1-yl)oxy)acetic acid (Compound (2)). The structure of compound (1), compound (2), the corresponding sodium salt (compound (3)), and the corresponding potassium salt (compound (4)) are provided in FIG. 1. Compound 2 is synthesized according to methods known to those of skill in the art, for example, in U.S. Pat. No. 6,831,186.

Compound (1) may be synthesized by standard techniques know to those of skill in the art. For example, Compound (1) may be synthesized from Compound (2) according to the following method:

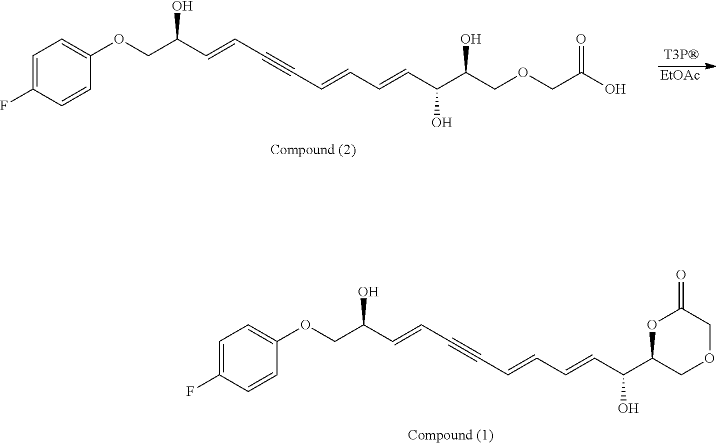 [SMILES: "EtOAc" ; O=C(O)COC[C@H](O)[C@H](O)/C=C/C=C/C#C/C=C/[C@H](O)COc1ccc(F)cc1 ; O=C1COC[C@@H]([C@H](O)/C=C/C=C/C#C/C=C/[C@H](O)COc2ccc(F)cc2)O1]

Compound (2) (884 mg, 1.989 mmol) was suspended in ethyl acetate (5 mL) in a 20 mL glass vial and a solution of T3P® (1-propanephosphonic acid cyclic anhydride) in ethyl acetate (5 ml, 3.97 mmol, 50% by weight, 2 eq.) was added dropwise. The mixture was stirred overnight at room temperature, then concentrated under reduced pressure. Purification of the residue by flash chromatography eluting with an increasing proportion of ethyl acetate in hexanes provided the title compound (287 mg, 37% yield) as a light yellow oil. ¹H NMR (CDCl₃, 400 MHz): δ 2.37 (1H, s), 2.48 (1H, s), 3.78-3.87 (2H, m), 3.93-4.00 (2H, m), 4.24 (1H, d, J=17.7 Hz), 4.39 (1H, d, J=17.7 Hz), 4.53 (2H, dd, J=3.1, 7.8 Hz), 4.61 (1H, s), 5.79 (2H, dd, J=3.4, 15.2 Hz), 6.08 (1H, t, J=1.9 Hz), 6.20 (1H, dd, J=5.4, 15.9 Hz), 6.46 (1H, dd, J=11.0, 15.1 Hz), 6.59 (1H, dd, J=11.0, 15.3 Hz), 6.82-6.85 (2H, m), 6.95-7.00 (2H, m). HPLC purity: 98.8%.

### Example 2. Compound (1) Activates Human GPR32 (RvD1 Receptor) in a Beta Arrestin Coupled System Assay

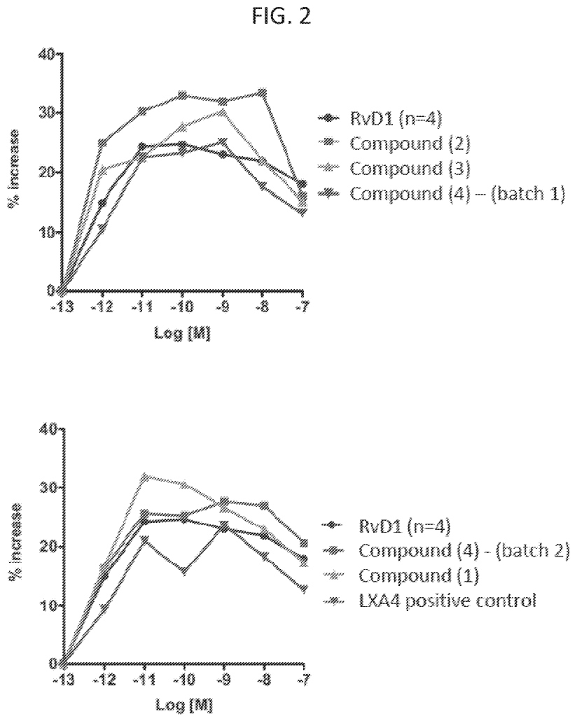

Ligand receptor interactions were monitored using the Beta-Arrestin PathHunter eXpress System™ (DiscoverX, Fremont, Calif.) and carried out with CHO cells stably overexpressing recombinant human GPR32 receptors. CHO-GPR32 cells were plated onto 96-well plates 24 h prior to experiments. Test compounds at indicated concentrations were incubated with cells for 1 h at 37° C. and receptor activation was determined by measuring chemiluminescence using the PathHunter detection kit. The results of this experiment are expressed as % increase of chemiluminescence above vehicle control (FIG. 2). Results are mean from 3 or 4 independent experiments and 3 replicates in each experiment.

### Example 3. Compound (1) Activates Human ALX/FPR2 (LXA₄ Receptor) in a Beta-Arrestin Coupled System Assay

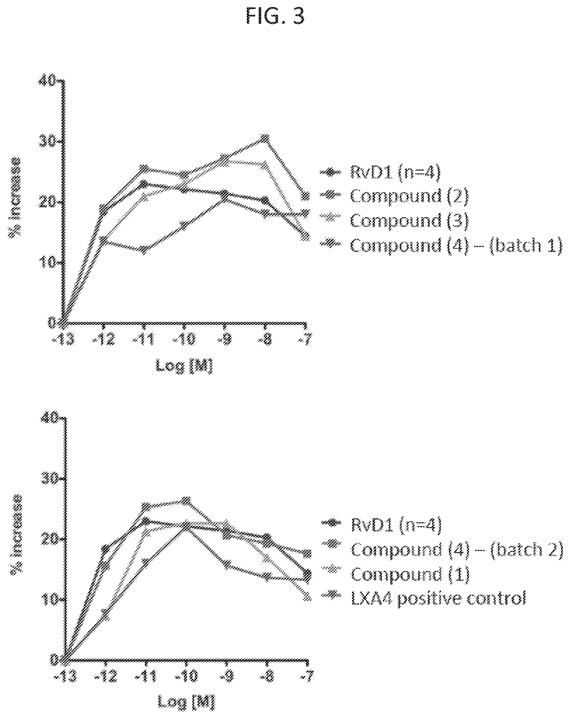

Ligand receptor interactions were monitored using the Beta-Arrestin PathHunter eXpress System™ (DiscoverX, Fremont, Calif.) and carried out with HEK cells stably overexpressing recombinant human ALX receptors. HEK-ALX cells were plated onto 96-well plates 24 h prior to experiments. Test compounds at indicated concentrations were incubated with cells for 1 h at 37° C. and receptor activation was determined by measuring chemiluminescence using the PathHunter detection kit. Results are expressed as % increase of chemiluminescence above vehicle control (FIG. 3). Results are mean from 3 or 4 independent experiments and 3 replicates in each experiment.

### Example 4. Compound (1), but not Compound (2), Binds to Cannabinoid Receptor 1 (CB₁) and Cannabinoid Receptor 2 (CB₂)

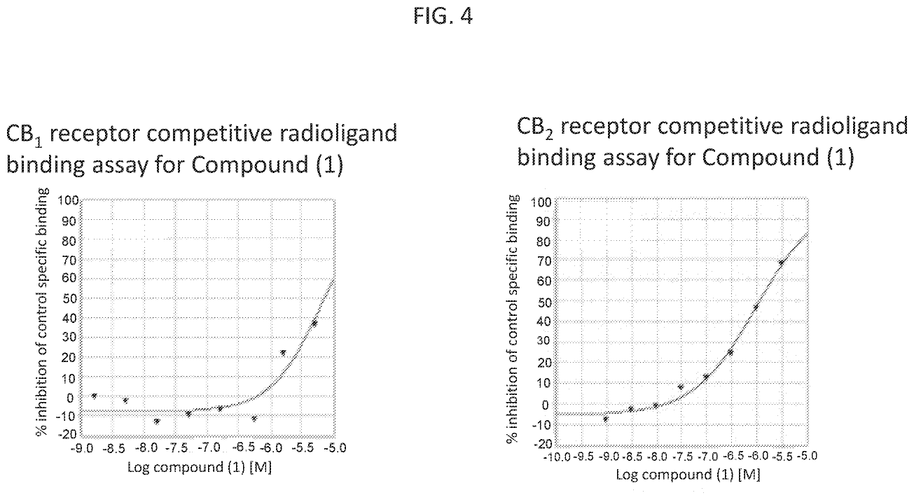

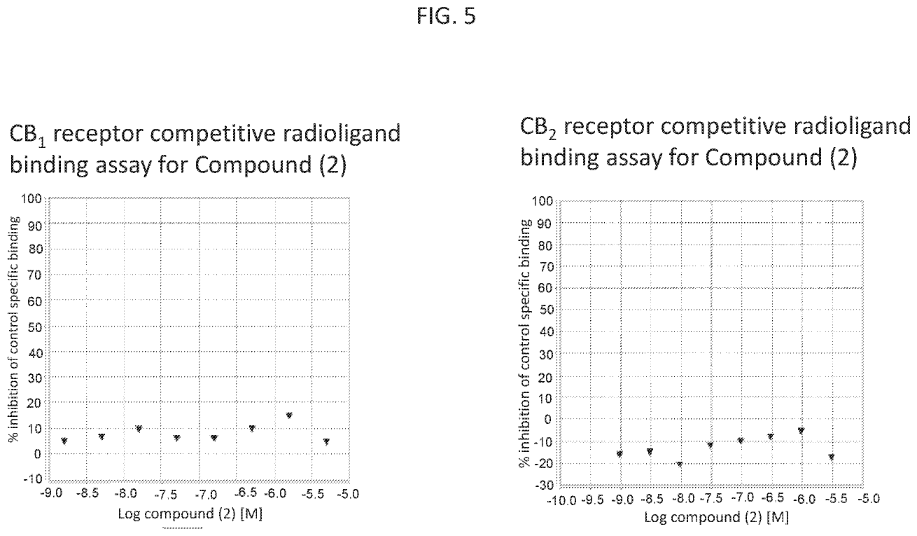

The binding affinity (% inhibition, K) of each of Compounds (1) and (2) for the CB₁ and CB₂ receptors was determined by a competitive radioligand binding assay. Compound (1) was observed to exhibit weak binding (K>5 μM) to the CB₁ receptor and bound to the CB₂ receptor with a K of 630 nM (FIG. 4). By comparison, no binding of the free acid to either the CB₁ or CB₂ receptors was observed (FIG. 5). The radioligand binding assays were performed as described below.

CB₁ Radioligand Binding Assay: Cell membrane homogenates (5 μg protein) prepared from Chem cells expressing human recombinant CB₁ receptor were incubated for 30 min at 22° C. with 2 nM [³H]CP 55940 (CB₁/CB₂ radioligand) in the absence or presence of the test compound in a buffer containing 50 mM Tris-HCl (pH 7.4), 5 mM MgCl₂, 2.5 mM EDTA and 0.3% BSA. Nonspecific binding is determined in the presence of 10 μM WIN 55212-2. Each compound was tested in 8 concentrations. For each concentration, % Inhibition was determined as a function of radioligand specific binding to the CB₁ receptor. The K was determined from the concentration-response curve of each tested compound.

CB₂ Radioligand Binding Assay: Cell membrane homogenates (12 μg protein) prepared from CHO cells expressing human recombinant CB₂ receptor were incubated for 120 min at 37° C. with 0.8 nM [³H]WIN 55212-2 (CB₁/CB₂ radioligand) in the absence or presence of the test compound in a buffer containing 50 mM Hepes/Tris (pH 7.4), 5 mM MgCl₂, 2.5 mM EGTA and 0.1% BSA. Nonspecific binding was determined in the presence of 5 μM WIN 55212-2. Each compound was tested in 8 concentrations. For each concentration, % Inhibition was determined as a function of radioligand specific binding to the CB₂ receptor. The K was determined from the concentration-response curve of each tested compound.

Further details for determining the binding affinity of compounds for a cannabinoid receptor by competitive radioligand binding can be found in the literature, for example, in Rinaldi-Carmona M, et al. Characterization of two cloned human CB₁ cannabinoid receptors isoform. J. Pharmacol. Exp. Ther. 278: 871 (1996); and Munro S., et al. Molecular characterization of a peripheral receptor for cannabinoids. Nature 365:61-65 (1993).

### Example 5. Compound (1) Enhances Human Macrophage Phagocytosis of Live E. coli

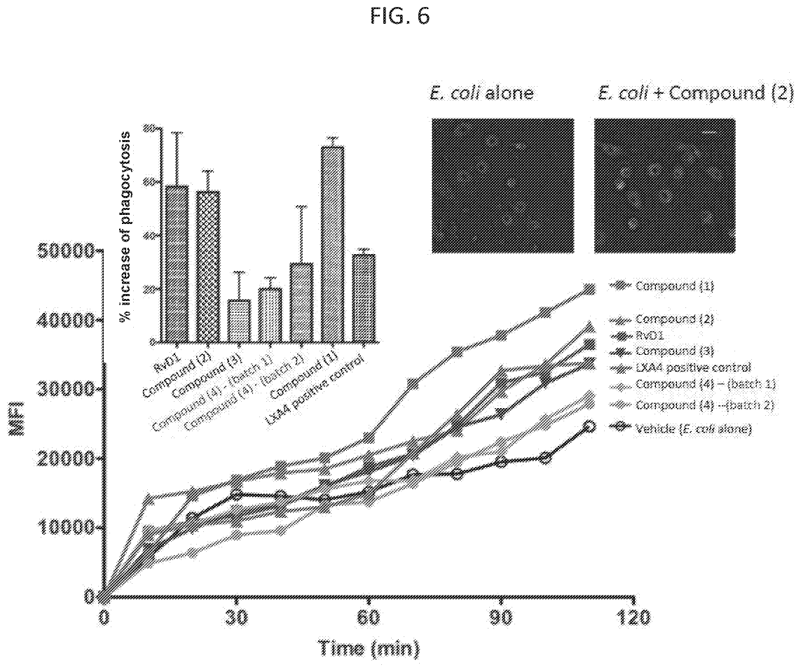

Human Mϕ were plated onto chamber slides (0.1×10⁶ cells/well), incubated with RvD1, compound (10 nM), or vehicle alone for 15 min at 37° C., followed by addition of BacLight Green-labeled E. coli to initiate phagocytosis. Fluorescent images were then recorded every 10 min for 120 min (37° C.) using Keyence BZ-9000 (BIOREVO) inverted fluorescence phase-contrast microscope (40× objective) equipped with a monochrome/color switching camera using BZ-II Viewer software (Keyence, Itasca, Ill., USA). Green fluorescence intensity was quantified using BZ-II Analyzer. Results are recordings of mean fluorescence intensity (MFI) from one representative donor (FIG. 6).

### Example 6. Compound (1) Enhances Human Macrophage Phagocytosis of Live E. coli: Dose Responses

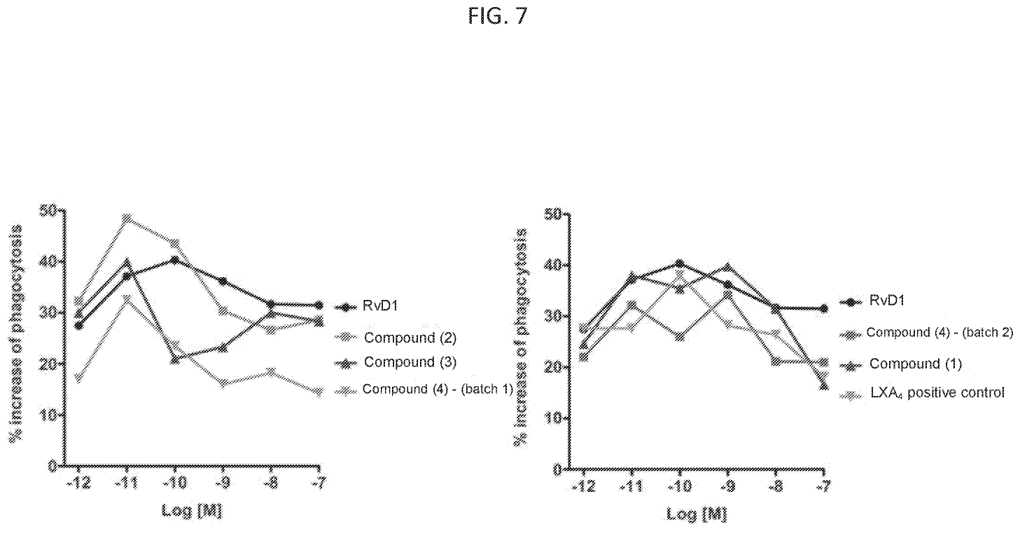

Human Mϕ were plated onto 96-well plates (5×10⁴ cells/well), incubated with RvD1, compound (1 pM-100 nM) or vehicle alone for 15 min at 37° C., followed by addition of BacLight Green-labeled E. coli (E. coli:Mϕ=50:1) to initiate phagocytosis. One hour later, fluorescence associated with Mϕ was determined using a microplate reader. Results are expressed as percent increase above vehicle (E. coli alone); mean from 4 separate experiments (FIG. 7).

### Example 7. Compound (1) Accelerates PMN Clearance In Vivo: E. coli Initiated Peritonitis with a Shorter T₅₀ as Compared to Compound (4)

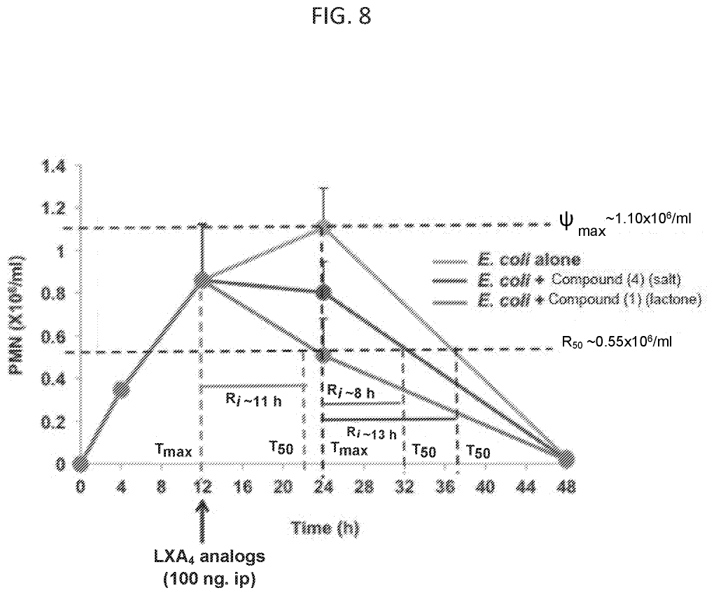

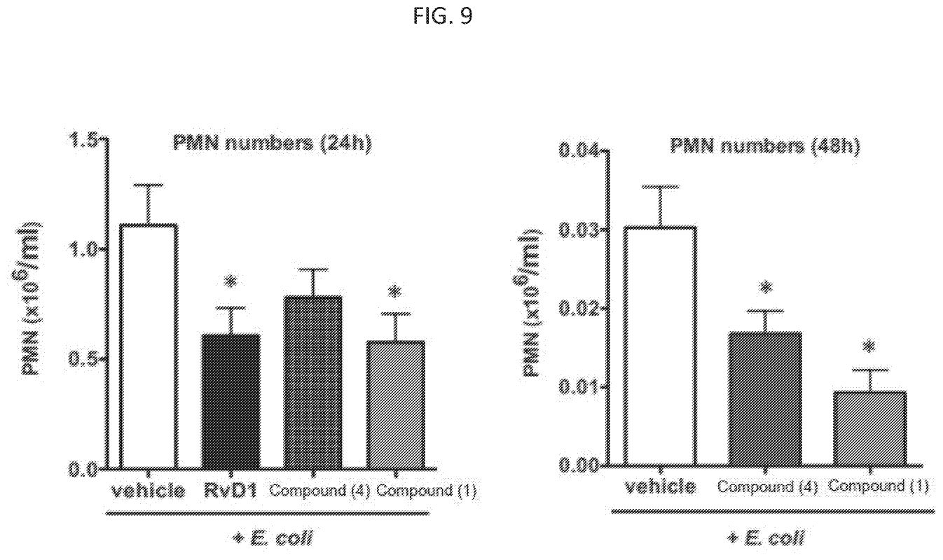

FVB mice (male, 6 wks old) were inoculated with E. coli (105 CFU). 100 ng of RvD1, compound (1), compound (4), or vehicle was given by intraperitoneal injection 12 h after E. coli inoculation, and peritoneal exudates collected at 24 and 48 hours. Total leukocytes were enumerated using light microscopy and PMN numbers were determined by flow cytometry. FIG. 8 shows the time course of PMN clearance. FIG. 9 shows the corresponding PMN numbers at 24 hours and 48 hours post-infection. Resolution indices were calculated using PMN numbers. As shown in Table 1, both compound (1) and compound (4) shortened the resolution interval (Ri), with compound (1) shifting the onset of resolution (Tmax) to an earlier time.

TABLE 1
PMN clearance in vivo

| | | | Resolution |
|---|---|---|---|
| | Tmax | T₅₀ | Interval |
| | (hours) | (hours) | (hours) |
| E. coli | 24 | 37 | 13 |
| E. coli + Compound (4) | 24 | 32 | 8 |
| E. coli + Compound (1) | 12 | 23 | 11 |

The length of time it takes for inflammation to peak (Tmax) and inflammatory resolution to reach 50% of maximum (T50) is a direct reflection of the ability of a drug to activate the resolution pathway. Compound (1) was most effective at reducing the Tmax of inflammation and Tso of resolution by shortening the time to resolve and clear infectious inflammation demonstrating that unlike anti-inflammatory agents is expected not to be immunosuppressive. Compound (1) unexpectantly, substantially reduced the Tmax by 50% and also reduced the Tso. Together these finding demonstrate that compound (1) administered in vivo was superior to the others tested in clearing and resolving infectious inflammation in vivo

### Example 8. Effect of Compound (1) on Cell Count in Mouse Peritonitis Model

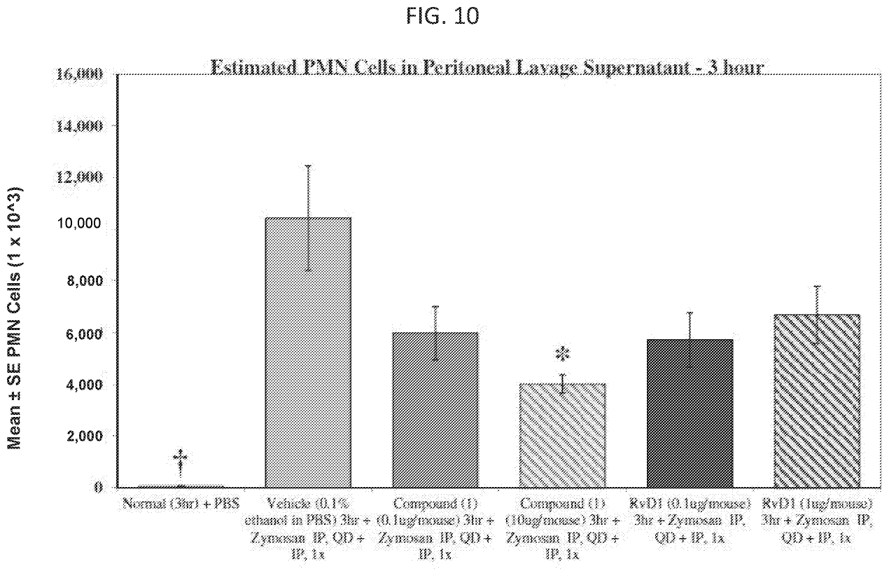

FVB mice (male, 6 wks old) were inoculated with E. coli (10⁵ CFU). 100 ng of RvD1, LXA₄ analogs (e.g., compound (1)) or vehicle was given by intraperitoneal injection 12 h after E. coli inoculation, and peritoneal exudates collected at 24 and 48 h. Total leukocytes were enumerated using light microscopy and PMN numbers were determined by flow cytometry. Table 2 shows the number of granulocytes, mononuclear cells, nucleated cells, and estimated PMNs following treatment with compound (1). FIG. 10 shows the mean absolute total nucleated cells in peritoneal lavage supernatant three hours post-administration of either 0.1 μg/mouse or 10 μg/mouse of compound (1).

TABLE 2
Effect of Compound (1) on cell count in mouse peritonitis model

| | | | | | |
|---|---|---|---|---|---|
| | | Granulocytes | Mononuclear | | |
| | | Counted | Counted | Nucleated Cells | Estimated PMNs |
| Group | Treatment | (100 cells) | (100 cells) | 10³/ml | 10³/ml |

3 h post-zymosan

| | | | | | | | | | |
|---|---|---|---|---|---|---|---|---|---|
| 1 | −Zymosan | \hat{ }2.6 | (0.8) | \hat{ }97.4 | (0.8) | \hat{ }1852.50 | (237.26) | \hat{ }54.53 | (21.41) |
| 2 | +Zymosan + Veh | 92.8 | (1.9) | 7.2 | (1.9) | 11145.00 | (1967.78) | 10425.38 | (2024.19) |
| 3 | +Zymosan + Cmpd(1) | 96.4 | (1.0) | 3.6 | (1.0) | 6165.00 | (1016.28) | 5977.50 | (1029.09) |
| | (0.1 μg/mouse) | | | | | | | | |
| 4 | +Zymosan + Cmpd(1) | 95.0 | (1.2) | 5.0 | (1.2) | *4237.50 | (414.54) | *4012.13 | (358.14) |
| | (10 μg/mouse) | | | | | | | | |
| 5 | +Zymosan + RvD1 | 89.8 | (3.7) | 10.8 | (3.4) | 6375.00 | (1050.40) | 5723.25 | (1045.36) |
| | (0.1 μg/mouse) | | | | | | | | |
| 6 | +Zymosan + RvD1 | 96.3 | (1.2) | 3.8 | (1.2) | 6232.50 | (1187.30) | 6685.13 | (1107.80) |
| | (1 μg/mouse) | | | | | | | | |

24 h post-zymosan

| | | | | | | | | | |
|---|---|---|---|---|---|---|---|---|---|
| 7 | −Zymosan | \hat{ }3.8 | (0.7) | \hat{ }96.2 | (0.7) | \hat{ }2932.50 | (668.24) | \hat{ }125.48 | (43.04) |
| 8 | +Zymosan + Veh | 92.4 | (1.2) | 7.6 | (1.2) | 16530.00 | (1778.56) | 15301.35 | (1701.04) |
| 9 | +Zymosan + Cmpd(1) | 88.8 | (2.7) | 11.2 | (2.7) | 16380.00 | (1261.61) | 14672.70 | (1514.49) |
| | (0.1 μg/mouse) | | | | | | | | |
| 10 | +Zymosan + RvD1 | 86.6 | (0.9) | 13.4 | (0.9) | 13267.50 | (2244.69) | 11496.23 | (1948.45) |
| | (1 μg/mouse) | | | | | | | | |

(SE) = Standard error displayed in parenthesis,
PMN = Polymorphonuclear leukocytes
Vehicle = 0.1% EtOH in PBS
*p < 0.05 ANOVA (Dunnett's post-hoc) vs. Vehicle control
\hat{ }p < 0.05 Student's t-test vs. Vehicle control

### Example 9. Effect of Compound (1) on Experimental Allergic Encephalomyelitis (EAE) Model

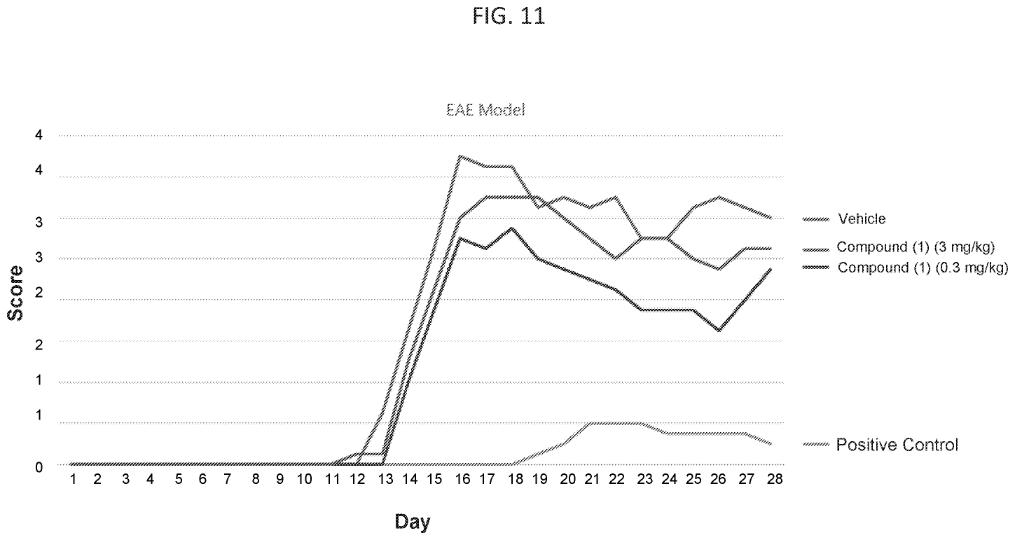

Mice (n=8/group) were anesthetized with isoflurane, and EAE induced by injecting 100 μL of a total emulsion containing 3 mg/mL MOG 35-55 and 8 mg/mL Complete Freund's Adjuvant in saline subcutaneously into the shaved backs of the mice at three sites: one along the midline of the back between the shoulders, and two on each side of the midline on the lower back. This day is recorded as day 0. Bordetella pertussis toxin (PTX, 200 ng in 200 μL of PBS) was administered i.p. on the day of immunization and 48 hours after for all groups. EAE development was assessed by clinically scoring of the mice once daily from Day 0 to Day 28 post immunization using the EAE Clinical Scoring System provided in Table 3. The corresponding results are provided in FIG. 11.

TABLE 3
EAE Clinical Scoring System

| Score | Clinical signs |
|---|---|
| 0 | Normal mouse; no overt signs of disease |
| 1 | Limp tail or hind limb weakness but not both |
| 2 | Limp tail and hind limb weakness |
| 3 | Partial hind limb paralysis |
| 4 | Complete hind limb paralysis |
| 5 | Moribund state; death by EAE: sacrifice for humane reasons |

### Example 10. Rat Pharmacokinetic Studies: Comparison of Compound (1) and Compound (2)

Sprague Dawley (SD) rats (n=3/group) were administered either compound (1) or compound (2) orally at 10 mg/kg (5% ethanol, 5% VE-TPGS, 90% PEG400) or intravenously at 0.3 mg/kg (5% NMP, 95% PEG400). The resulting individual and mean plasma concentration-time was measured for the free acid, compound (2), with the data provided in Tables 4-7. A comparison of the calculated pharmacokinetic (PK) parameters for compounds (1) and (2) is provided in Table 8.

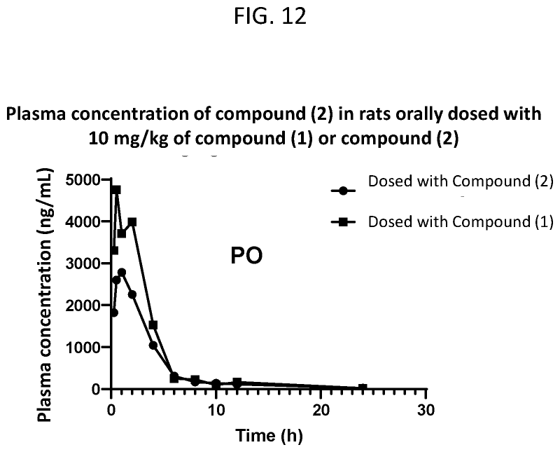

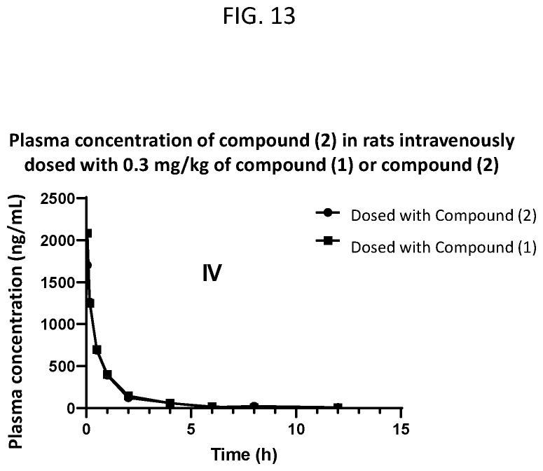

In particular, the inventors have observed that the lactone-containing compound (1) exhibits and increased maximum peak plasma drug concentration (Cₘₐₓ), an increased area under the plasma concentration-time curve from time zero to time of last measurable concentration (AUCₗₐₛₜ), and an increased % bioavailability (F), following oral administration of 10 mg/kg to SD rats, as compared to the corresponding free acid compound (2). Particularly notable is the increase in % oral bioavailability of approximately 45%. The plasma concentration of compound (2) following oral administration of either compound (1) or compound (2) in rat orally dosed with 10 mg/kg is provided in FIG. 12. An increase in the AUCₗₐₛₜ upon IV administration of 0.3 mg/kg was also observed. The plasma concentration of compound (2) following oral administration of either compound (1) or compound (2) in rat IV dosed with 0.3 mg/kg is provided in FIG. 13. This suggests that compound (1) has improved PK performance as compared to compound (2), in particular with regards to oral bioavailability.

TABLE 4
Individual and mean plasma concentration-time data of compound
(2) following an oral dose of 10 mg/kg compound (1) in SD rats

| | | Sampling | Concentration | | | | | |
|---|---|---|---|---|---|---|---|---|
| Dose | Dose | time | (ng/mL) | | | Mean | Standard | |
| (mg/kg) | route | (hr) | Individual | | | (ng/mL) | Deviation | CV(%) |
| 10 | PO | 0.25 | 4060 | 2011 | 3844 | 3305 | 1126 | 34.1 |
| | Compound | 0.5 | 6108 | 2755 | 5405 | 4756 | 1768 | 37.2 |
| | (1) | 1 | 4096 | 3269 | 3766 | 3710 | 416 | 11.2 |
| | | 2 | 2854 | 3385 | 5718 | 3986 | 1524 | 38.2 |
| | | 4 | 610 | 2182 | 1796 | 1529 | 819 | 53.6 |
| | | 6 | 261 | 253 | 239 | 251 | 11.3 | 4.49 |
| | | 8 | 125 | 327 | 235 | 229 | 101 | 44.3 |
| | | 10 | 108 | 78.4 | 107 | 97.7 | 16.7 | 17.1 |
| | | 12 | 111 | 247 | 141 | 166 | 71.7 | 43.1 |
| | | 24 | 24.1 | 24.0 | 13.6 | 20.6 | 6.00 | 29.2 |

TABLE 5
Individual and mean plasma concentration-time data of compound
(2) following an IV dose of 0.3 mg/kg compound (1) in SD rats

| | | Sampling | Concentration | | | | | |
|---|---|---|---|---|---|---|---|---|
| Dose | Dose | time | (ng/mL) | | | Mean | Standard | |
| (mg/kg) | route | (hr) | Individual | | | (ng/mL) | Deviation | CV(%) |
| 0.3 | IV | 0.05 | 1645 | 2677 | 1934 | 2085 | 532 | 25.5 |
| | Compound | 0.167 | 1064 | 1303 | 1374 | 1247 | 162 | 13.0 |
| | (1) | 0.5 | 690 | 637 | 761 | 696 | 62.0 | 8.91 |
| | | 1 | 289 | 475 | 436 | 400 | 98.4 | 24.6 |
| | | 2 | 128 | 133 | 173 | 145 | 24.2 | 16.7 |
| | | 4 | 51.6 | 52.2 | 68.7 | 57.5 | 9.70 | 16.9 |
| | | 6 | 15.9 | 14.4 | 13.7 | 14.6 | 1.13 | 7.74 |
| | | 8 | BQL | BQL | 5.71 | 5.71 | NA | NA |
| | | 12 | BQL | 5.14 | 6.14 | 5.64 | NA | NA |
| | | 24 | BQL | BQL | BQL | BQL | NA | NA |

TABLE 6
Individual and mean plasma concentration-time data of compound
(2) following an oral dose of 10 mg/kg compound (2) in SD rats

| | | Sampling | Concentration | | | | | |
|---|---|---|---|---|---|---|---|---|
| Dose | Dose | time | (ng/mL) | | | Mean | Standard | |
| (mg/kg) | route | (hr) | Individual | | | (ng/mL) | Deviation | CV(%) |
| 10 | Oral | 0.25 | 1902 | 1725 | 1836 | 1821 | 89.4 | 4.91 |
| | Compound | 0.5 | 2386 | 2491 | 2926 | 2601 | 286 | 11.0 |
| | (2) | 1 | 2602 | 2673 | 3077 | 2784 | 256 | 9.20 |
| | | 2 | 2199 | 1758 | 2808 | 2255 | 527 | 23.4 |
| | | 4 | 1446 | 1084 | 602 | 1044 | 424 | 40.6 |
| | | 6 | 433 | 157 | 339 | 310 | 140 | 45.2 |
| | | 8 | 197 | 184 | 129 | 170 | 35.9 | 21.2 |
| | | 10 | 220 | 84.3 | 110 | 138 | 72.1 | 52.2 |
| | | 12 | 123 | 126 | 93.8 | 114 | 17.9 | 15.6 |
| | | 24 | 31.0 | 9.33 | 28.3 | 22.9 | 11.8 | 51.6 |

TABLE 7
Individual and mean plasma concentration-time data of compound
(2) following an IV dose of 0.3 mg/kg compound (2) in SD rats

| | | Sampling | Concentration | | | | | |
|---|---|---|---|---|---|---|---|---|
| Dose | Dose | time | (ng/mL) | | | Mean | Standard | |
| (mg/kg) | route | (hr) | Individual | | | (ng/mL) | Deviation | CV(%) |
| 0.3 | IV | 0.05 | 1688 | 1660 | 1763 | 1704 | 53.3 | 3.13 |
| | Compound | 0.167 | 1125 | 1237 | 1428 | 1263 | 153 | 12.1 |
| | (2) | 0.5 | 571 | 701 | 792 | 688 | 111 | 16.2 |
| | | 1 | 325 | 366 | 479 | 390 | 79.5 | 20.4 |
| | | 2 | 91.5 | 117 | 154 | 121 | 31.6 | 26.1 |
| | | 4 | 52.0 | 56.7 | 68.2 | 58.9 | 8.33 | 14.1 |
| | | 6 | 5.26 | 9.74 | 11.1 | 8.71 | 3.06 | 35.2 |
| | | 8 | 39.9 | 10.7 | 15.7 | 22.1 | 15.6 | 70.7 |
| | | 12 | BQL | 8.33 | 5.02 | 6.67 | NA | NA |
| | | 24 | BQL | BQL | BQL | BQL | NA | NA |

TABLE 8
Comparison of PK parameters for compound (1) and compound (2)

| | | Compound (1) | | Compound (2) | |
|---|---|---|---|---|---|
| | | PO (10 | IV (0.3 | PO (10 | IV (0.3 |
| PK parameter | Unit | mg/kg) | mg/kg) | mg/kg) | mg/kg) |
| CL | mL/min/kg | | 3.31 | | 3.41 |
| Vss | L/kg | | 0.26 | | 0.32 |
| Tₘₐₓ | hr | 0.5 | | 1.00 | |
| Cₘₐₓ | ng/mL | 4756 | | 2784 | |
| t_{1/2} | hr | 4.99 | 1.45 | 5.32 | 1.50 |
| AUCₗₐₛₜ | hr*ng/mL | 16872 | 1499 | 11162 | 1450 |
| F | % | 33.8 | | 23.2 | |

### OTHER EMBODIMENTS

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

What is claimed is:

1. A pharmaceutical composition comprising a therapeutically effective amount of compound (1) and a pharmaceutically acceptable excipient, wherein compound (1) has the structure:

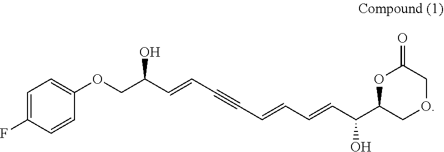 [SMILES: O=C1COC[C@@H]([C@H](O)/C=C/C=C/C#C/C=C/[C@H](O)COc2ccc(F)cc2)O1]

2. A method for treating an inflammatory disorder in a subject in need thereof, the method comprising administering to the subject the pharmaceutical composition of claim 1.

3. The method of claim 2, wherein the inflammatory disorder is an autoimmune disorder.

4. The method of claim 2, wherein the inflammatory disorder is scleroderma, dermatomyositis, systemic lupus erythematosus, periodontitis, an inflammatory bowel disease (IBD), chronic obstructive pulmonary disease (COPD), acquired immune deficiency syndrome (AIDS), multiple sclerosis, rheumatoid arthritis, psoriasis, diabetes, cancer, asthma, atopic dermatitis, an autoimmune thyroid disorder, stroke, ischemia, amyotrophic lateral sclerosis (ALS), chronic traumatic encephalopathy (CTE), chronic inflammatory demyelinating polyneuropathy, hepatitis, autoimmune inner ear disease, uveitis, iritis, peritonitis, or a neurodegenerative disease.

5. The method of claim 4, wherein the inflammatory bowel disease is ulcerative colitis or Crohn's disease.

6. The method of claim 4, wherein the diabetes is type 1 diabetes.

7. The method of claim 4, wherein the neurodegenerative disorder is Alzheimer's disease or Parkinson's disease.

8. The method of claim 4, wherein the autoimmune thyroid disorder is Graves' disease or Hashimoto's disease.

9. A method for treating a fibrotic disorder in a subject in need thereof, the method comprising administering to the subject the pharmaceutical composition of claim 1.

10. The method of claim 9, wherein the fibrotic disorder is cystic fibrosis, scleroderma, liver cirrhosis, interstitial pulmonary fibrosis, idiopathic pulmonary fibrosis, Dupuytren's contracture, keloids, chronic kidney disease, chronic graft rejection, scarring or wound healing abnormalities, post-operative adhesions, reactive fibrosis, polymyositis, ANCA vasculitis, Behçet's disease, anti-phospholipid syndrome, relapsing polychondritis, familial Mediterranean fever, giant cell arteritis, Graves' ophthalmopathy, discoid lupus, pemphigus, bullous pemphigoid, hydradenitis suppuritiva, sarcoidosis, bronchiolitis obliterans, interstitial lung disease, primary sclerosing cholangitis, primary biliary cirrhosis, arterial stiffness, mediastinal fibrosis, Peyronie's disease, nephrogenic systemic fibrosis, adhesive capsulitis, dermal fibrosis, lung fibrosis, liver fibrosis, kidney fibrosis, brain fibrosis, and heart fibrosis.

11. The method of claim 4 or 10, wherein the scleroderma is systemic sclerosis, sine scleroderma, or a scleroderma-like disorder.

12. The method of any one of claims 1-11, wherein the pharmaceutical composition is administered parenterally, orally, topically, nasally, rectally, buccally, by ophthalmic administration, or by inhalation.

13. The method of claim 12, wherein the parenteral administration is intraperitoneal, subcutaneous, intracutaneous, intravenous, intramuscular, intraarticular, intraarterial, intrasynovial, intrasternal, intrathecal, intralesional, intracranial, or intracardiac.

14. The method of any one of claims 1-12, wherein the pharmaceutical composition is in the form of a tablet or a capsule.

15. The pharmaceutical composition of claim 14, wherein the pharmaceutical composition comprises an enteric coating.

16. The method of any one of claims 1-12, wherein the pharmaceutical composition is in the form of a solution, an ointment, a cream, a suspension, a lotion, a powder, a paste, a gel, a spray, an aerosol, or an oil.

17. The method of any one of claims 1-16, wherein the pharmaceutical composition is formulated for sustained release.

18. The method of any one of claims 1-17, wherein the composition is administered once daily, twice daily, or three times daily.

19. The method of any one of claims 1-17, wherein the composition is administered once a week, twice a week, once a month, or twice a month.

20. The method of any one of claims 1-19, wherein the method further comprises administering the pharmaceutical composition in combination with an additional therapeutic agent.

21. The method of claim 20, wherein the additional therapeutic agent is an anti-inflammatory agent.

22. The method of claim 21, wherein the anti-inflammatory agent is a nonsteroidal anti-inflammatory drug (NSAID).

23. The method of claim 22, wherein the NSAID is acetylsalicylic acid, celecoxib, rofecoxib, valdecoxib, diclofenac, diflunisal, etodolac, ibuprofen, flurbiprofen, indomethacin, ketoprofen, ketorolac, nabumetone, naproxen, oxaprozin, piroxicam, sulindac, tolmetin, meclofenamate, mefenamic acid, or meloxicam.

24. The method of claim 20, wherein the additional therapeutic agent is a corticosteroid.

25. The method of claim 24, wherein the corticosteroid is algestone, 6-alpha-fluoroprednisolone, 6-alphamethylprednisolone, 6-alpha-methylprednisolone 21-acetate, 6-alpha-methylprednisolone 21-hemisuccinate sodium salt, 6-alpha,9-alpha-difluoroprednisolone 21-acetate 17-butyrate, amcinafal, beclomethasone, beclomethasone dipropionate, beclomethasone dipropionate monohydrate, 6-beta-hydroxycortisol, betamethasone, betamethasone-17-valerate, budesonide, clobetasol, clobetasol propionate, clobetasone, clocortolone, clocortolone pivalate, cortisone, cortisone acetate, cortodoxone, deflazacort, 21-deoxycortisol, deprodone, descinolone, desonide, desoximethasone, dexamethasone, dexamethasone-21-acetate, dichlorisone, diflorasone, diflorasone diacetate, diflucortolone, doxibetasol, fludrocortisone, flumethasone, flumethasone pivalate, flumoxonide, flunisolide, fluocinonide, fluocinolone acetonide, 9-fluorocortisone, fluorohydroxyandrostenedione, fluorometholone, fluorometholone acetate, fluoxymesterone, fluprednidene, fluprednisolone, flurandrenolide, formocortal, halcinonide, halometasone, halopredone, hyrcanoside, hydrocortisone, hydrocortisone acetate, hydrocortisone butyrate, hydrocortisone cypionate, hydrocortisone sodium phosphate, hydrocortisone sodium succinate, hydrocortisone probutate, hydrocortisone valerate, 6-hydroxydexamethasone, isoflupredone, isoflupredone acetate, isoprednidene, meclorisone, methylprednisolone, methylprednisolone acetate, methylprednisolone sodium succinate, paramethasone, paramethasone acetate, prednisolone, prednisolone acetate, prednisolone metasulphobenzoate, prednisolone sodium phosphate, prednisolone tebutate, prednisolone-21-hemisuccinate free acid, prednisolone-21-acetate, prednisolone-21-(beta-D-glucuronide), prednisone, prednylidene, procinonide, tralonide, triamcinolone, triamcinolone acetonide, triamcinolone acetonide 21-palmitate, triamcinolone diacetate, triamcinolone hexacetonide, or wortmannin.
